(19) United States
(12) Patent Application Publication
(10) Pub. No.: US 2016/0376592 A1
(43) Pub. Date: Dec. 29, 2016

(54) Methods of Manipulating the Fate of Cells

(71) Applicant: Ivan Bergstein, New York, NY (US)

(72) Inventor: Ivan Bergstein, New York, NY (US)

(21) Appl. No.: 15/263,458

(22) Filed: Sep. 13, 2016

Related U.S. Application Data
(63) Continuation of application No. 14/635,368, filed on Mar. 2, 2015, now abandoned.
Application No. 14/635,368 is a continuation of application No. 14/064,498, filed on Oct. 28, 2013, now abandoned.
Application No. 14/064,498 is a continuation of application No. 10/849,037, filed on May 20, 2004, now abandoned.
Application No. 10/849,037 is a continuation of application No. 10/177,886, filed on Jun. 21, 2002, now abandoned.
(60) Provisional application No. 60/300,389, filed on Jun. 22, 2001.

(51) Int. Cl. C12N 15/113; A61K 31/713; C12Q 1/68; A61K 45/06

(52) U.S. Cl. CPC C12N 15/113; A61K 45/06; A61K 31/713; C12Q 1/6886; C12N 2310/113; C12N 2320/31; C12Q 2600/178; C12Q 2600/136

(57) ABSTRACT

A method of manipulating the fate of a cell, which comprises contacting the cell with at least one of (a) a cell fate-determining untranslated/noncoding RNA species (cuR), (b) a modified cuR, or (c) a compound that modifies or affects cuR, under conditions sufficient to cause a cell-changing or cell-maintaining fate that results in cell regeneration, cell differentiation or cell death, so that an increase of desirable cells or a decrease in undesirable cells can be obtained. Another aspect of the invention relates to a method of manipulating the fate of a cell by contacting the cell with a compound that affects a fate-determining mechanism involving homologous nucleic acid interactions of RNA:RNA or RNA:DNA or resolution of such interactions under conditions sufficient to cause a cell-changing or cell-maintaining fate that results in cell regeneration, cell differentiation or cell death, so that an increase of desirable cells or a decrease in undesirable cells can be obtained. The invention generates cell fate or cell maintenance in a subject, such as a human, so that an increase of desirable cells or a decrease in undesirable cells can be obtained in the subject. This feature can be applied to a therapeutic method of treating a condition in a subject.

### CROSS-REFERENCE TO RELATED APPLICATION

This application claims priority under 35 USC §119(e) (1) of provisional application No. 60/300,389, filed Jun. 22, 2001.

### FIELD OF THE INVENTION

The present invention relates to a method of manipulating the cellular fate-determining mechanisms of cancer cells, stem cells, and certain stem cell progeny. More specifically, the present invention relates to a method of treating cancer, wherein cancer stem cells and progeny cells can be specifically targeted based on a specific fate-determining mechanism. The invention also encompasses a method of manipulating the fate-determining mechanisms to expand or contract cell pools for purposes of studying or treating conditions relating to regenerative medicine or developmental biology. The present invention further relates to a method of isolating or screening for molecules involved in the fate-determining mechanism. The present invention further relates to a method of designing a compound or screening for a compound that interacts with the identified fate-determining molecule, and also relates to a compound found to manipulate a fate-determining mechanism.

### BACKGROUND OF THE INVENTION

Classic cancer therapies, such as, chemotherapy and radiation, work primarily by eradicating tumor cells which are fast-growing or highly proliferative. More recent anti-cancer strategies have attempted to eradicate these same cells by more specific targeting of their antigenic and mutational alterations via immunotherapy and gene therapy. The present tactics, however, failed to target or eradicate a key cancer cell population, termed the cancer “stemline”, which is largely slow-growing or quiescent and without the antigenic and mutational alterations expressed by fast-growing cancer cells.

To date, regenerative biology has concentrated on the expansion and subsequent directed differentiation of stem cells and their maturing progeny through use of cytokines, ligands, receptors, and manipulation of cell signaling pathways. A need exists for the ability to manipulate the fate of cells to increase or decrease cell pools and to properly treat regenerative diseases.

The present invention addresses these needs and discloses methods of manipulating the fate-determining mechanisms, as well as method used for identifying cuRs and compounds affecting cuRs.

### SUMMARY OF THE INVENTION

The invention relates to a method of increasing desirable cell population or decreasing undesirable cell population which comprises manipulating the fate of a cell through contact with a compound that affects a cell fate-determining mechanism involving RNA-silencing, RNA-interference, RNA-editing, snoRNA-mediated modifications, tRNA-primed events, or other fate-determining mechanism involving homologous nucleic acid interactions of RNA:RNA, RNA:DNA, or DNA:DNA and their resolution under conditions to cause the cell to change or maintain fate.

Typically the method is used for applications involving regenerative biology, medicine, developmental biology, cancer, and aging causing cell regeneration, cell differentiation, or cell death. Advantageously, the cell is a stem cell, regenerative cell, or a cancer cell and the compound is naturally occurring, synthesized, or procured through a screen and comprises a nucleic acid, a protein, a riboprotein, a vaccine, a small molecule, or a chemical compound. Preferably the compound is identified through use of an assay in a screen of biological or chemical libraries or the compound is intelligently designed.

In one embodiment of the invention the compound the compound affects a cell fate-determining untranslated/noncoding RNA species (cuR), or a cuR-binding DNA region. The cuR is preferably single-stranded, double-stranded, part of an RNA:DNA complex, or part of a riboprotein complex. In yet another embodiment of the invention the compound affects a cuR-associated protein, a cuR-associated riboprotein, or other nucleic acid, protein or riboprotein involved in the cell fate-determining mechanism. Most preferably the protein or riboprotein affected is qde1, sgs2, ego1, qde2, rde1, qde3, mut7, piwi, hiwi, MRP, or ADAR.

The invention can be carried out by using a compound that modifies the cuR by RNA-silencing, RNA-interference, RNA-editing, or snoRNA-mediated modification. The modifications can include the production of small interfering RNA, small temporal RNA, microRNA, aberrant RNA, edited RNA, guide RNA, tRNA, tRNA-like species, snoRNA, snoRNA-like species, retroelement-derived RNA, imprinted RNA, allelically excluded RNA, or other modified or noncoding RNA. It is most preferably that the cuR affected be one of the following: Tsix, Xist, a switch (S) sequence, let-7, lin-4. kappaNE, kappaBS, Disc1, Disc2, C6orf4-6, C6UAS, Okazaki RNA, tRNA, tRNA-like species, snoRNA, snoRNA-like species, Air RNA, BC1 RNA, mir 142, or noncoding RNA within regions i(12p), 11q13, 11p, 15q.

Most preferably the compound is administered to a human in order to increase desirable cell population or decrease undesirable cell population therein. Advantageously, the compound can be administered in combination with another related compound, or agent including chemotherapy, radiation, differentiation, immunotherapy, gene therapy, cancer therapy, or regenerative therapy. Preferably the the administration of the compound causes a level of toxicity which is clinically tolerable.

A variation of the invention relates to a method of manipulating the fate of a cell, which comprises contacting the cell with at least one of (a) a cuR, (b) a modified cuR, or (c) a compound that modifies or affects cuR, under conditions sufficient to cause a cell-changing or cell-maintaining fate that results in cell regeneration, cell differentiation or cell death, so that an increase of desirable cells or a decrease in undesirable cells can be obtained.

As noted above, the cell is a stem cell or a cancer cell and the compound is a nucleic acid, a protein, a riboprotein, a vaccine, a small molecule or a chemical compound that changes cuR properties or function. Preferably, the compound includes a cuR, a cuR-binding DNA sequence, a modified cuR, a cuR-associated protein, or a cuR-riboprotein, and most preferably a sense-cuR or anti-cuR. The cuR may any of the specific entities disclosed herein. Furthermore, the cuR can act to epigenetically modify or alter DNA and the altered or modified DNA causes the fate change or fate maintenance of the cell.

When the invention is used to generate cell fate or cell maintenance in a human, the compound can be administered orally, intravenously, intradermally, intraperitoneally, subcutaneously, or rectally either alone or in combination with another therapy including chemotherapy, radiation, differentiation therapy, immunotherapy, or gene therapy. As the compound to be administered causes a level of toxicity which is clinically tolerable, it is possible to successfully treat conditions such as cancer or a degenerative disease.

Another aspect of the invention relates to a method of manipulating the fate of a cell by contacting the cell with a compound that affects a fate-determining mechanism involving homologous nucleic acid interactions of RNA:RNA or RNA:DNA or resolution of such interactions under conditions sufficient to cause a cell-changing or cell-maintaining fate that results in cell regeneration, cell differentiation or cell death, so that an increase of desirable cells or a decrease in undesirable cells can be obtained. Here, the cell can be a stem cell or a cancer cell and the compound is a nucleic acid, a protein, a riboprotein, a vaccine, a small molecule or a chemical compound that affects the resolution of a homologous nucleic acid interaction. Again, the method can be carried out in a subject, preferably a human subject, so that an increase of desirable cells or a decrease in undesirable cells can be obtained in the subject. In this method, the homologous nucleic acid interactions are preferably resolved in general by proteins or riboproteins and in particular by qde1; sgs2; ego1; qde2; rde1; qde3; mut-7; piwi; hiwi; RMRP; or ADAR.

Yet another embodiment of the invention relates to a method of procuring a cell fate-determining substance from a stem cell or progenitor cell for use as a target or a compound which comprises: purifying stem cells or progenitor cells from a larger population of cells; isolating cuRs which includes modified cuRs from the stem cell or progenitor cells, optionally with amplification, if necessary, to form an isolate; comparing the isolate with cuRs of non-stem cells to identify stem cell-specific cuRs; and identifying the stem cell-specific cuRs by one of: (a) cloning and sequencing; (b) screening the stem cell-specific cuRs for cell fate maintaining or cell fate changing activity through use of an assay system; or (c) using the stem cell-specific cuRs as probes to screen a library of compounds for complementarity or cell fate maintaining or cell fate changing activity through use of an assay system. Most preferably the stem cell from which the substance is purified is cancer stem cells which are purified from blood, bone marrow, or tissues of patients, from which the cuRs are isolated, and if necessary amplified. The cuRs are then compared by subtraction or differential analysis with cuRs from non-stem cells and cloned. A library of cuRs is then screened for activity and the library of cuRs is used to screen a cDNA array or other collection of nucleic acids, or the cuRs are sequenced and identified.

A variation of this method includes procuring a cell fate-determining substance from a stem cell which comprises purifying one or more stem cells from a larger population of cells; isolating one or more cuR(s) from the stem cell(s), optionally with amplification, if necessary, to form an isolate; comparing the isolate with cuRs of non-stem cells to identify stem cell specific cuRs; and screening the stem cell specific cuRs for cell fate maintaining or cell fate changing activity. These methods can further comprise identifying a compound that interacts with the stem cell specific cuRs by contacting the stem cell specific cuR(s) with a biological or chemical library of nucleic acids, proteins, chemical compounds or small molecules in order to identify the interactive compound. Thus, a compound that interacts with the stem cell specific cuRs can be intelligently designed based on the identification of the stem cell specific cuR(s).

Yet another embodiment of the invention is a method of decreasing undesirable cell populations, as for cancer treatment, which comprises manipulating the mechanism involving RNA-silencing, RNA-interference, RNA-editing, snoRNA-mediated modifications, tRNA-primed events, and other homologous nucleic acid interactions of RNA:RNA, RNA:DNA, DNA:DNA and their resolution under conditions to cause the cell to change fate by virtue of activating parasitic elements including endogenous viruses, transposes, and related selfish elements.

### Discussion of Biological Bases of Invention

### Untranslated/Noncoding RNA-Mediated RNA:RNA and RNA:DNA Interactions

Stem cells and certain stem cell progeny of higher eukaryotes, including humans, produce daughter cells which do not change fate and some daughter cells which do change fate and differentiate. This process is mechanistically similar to the asymmetric mitotic division program utilized by lower eukaryotes, such as yeast, nematodes, fruit flies, during development (1-4). Specifically, the cell fate mechanisms enacted by certain yeast is highlighted particularly by an interallelic communication event at the mating type locus (MAT) solely in the daughter that will change fate but not in the daughter of unchanged fate (1, 5). This event involves DNA:DNA pairing between homologous allelic sequences and resolution thereof via DNA/genetic recombination—a process that results in silencing of one of the 2 interacting previously active alleles. This program then triggers a cascade of events leading ultimately to a cell fate change (1, 5). Higher eukaryotic stem cells and possibly certain of their maturing non-stem cell progeny utilize a mechanistically related program to direct cell fate transitions, specifically involving DNA:DNA pairing and recombination.

Interestingly, however, there is evidence that the aforementioned interallelic events of yeast, involving DNA:DNA pairing resolved by genetic recombination, may constitute but one specific example of a more general phenomenon wherein homologous sequences can communicate not only at the DNA level (e.g. DNA:DNA pairing), but also at the RNA level (e.g. RNA:RNA pairing), or a combination level (e.g. RNA:DNA pairing)—and be resolved by both genetic (e.g. DNA:DNA-mediated recombination) as well as epigenetic means, such as DNA:DNA-mediated exchange of methylation/chromatin/nucleosomal structure or post-transcriptional RNA:RNA-mediated RNA degradation or translation inhibition.

For example, certain fungi resolve or silence an allelic participant in a homologous DNA:DNA interaction via exchange not of DNA but of epigenetic, for example, methylation, structure (7). That a related phenomenon occurs in human cells is revealed by evidence that certain homologous loci can indeed undergo DNA:DNA pairing (8), and at times withstand inter-allelic exchange of epigenetic structure (e.g. methylation pattern) (9, 10). Other organisms resolve certain homologous RNA:RNA events, by post-transcriptional double-stranded RNA (dsRNA) mediated RNA degradation (11, 12). Of note, it has been suggested that related post-transcriptional RNA-level processes may direct more permanent transcriptional level changes, via RNA:DNA-mediated events, or more specifically via RNA- or RNA:RNA-mediated changes in DNA methylation, which can then be more reliably perpetuated (13-16).

Indeed, a variety of once-thought-to-be-unique and disparate processes involving homologous sequence interactions at the DNA and RNA levels have been recently shown to be mechanistically related (17-22). Homologous DNA:DNA, RNA:RNA, or RNA:DNA interactions generally termed “cosuppression,” normally function in a wide range of eukaryotic organisms including plants, fungi, nematodes, fruit flies, simple vertebrates, and mammals, specifically as a primitive defense system against potentially fate-threatening sequences of parasitic origin, those, which are virus-, transposon-, or transgene-derived (17-22).

Mechanistically, “foreign/parasitic-appearing” DNA and RNA take the form specifically of paired nucleic acid structures, such as, DNA repeats or double-stranded RNA (dsRNA). Such complexes often form as intermediates in the replication-propagation life cycle of certain virus or transposon parasite-derived sequences, are structures that can be sensed and silenced (e.g. via DNA methylation or dsRNA-mediated RNA degradation or translation inhibition) by host cells attempting to limit parasite-related threats to cell integrity. In this way, the defense program constitutes a well-conserved protective mechanism by which host cells can maintain their fate against sequences sensed as potentially fate-threatening. Of note, it appears that the defense mechanism may be particularly important to those cell types that are most long-lived, as evidenced by the utilization of cosuppression-type activities by long-lived cells of the germline against fate-threatening transposable elements (23, 24).

That other long-lived cell types such as stem cells utilize similar fate-maintaining protective mechanisms characterized by the dismantling of potentially fate-altering paired structures, such as, homologous DNA:DNA, RNA:RNA, or RNA:DNA complexes, not only of parasitic-derivation but also ones which are native-borne, is a proposed new mechanism which, as will be demonstrated herein, has considerable support. In other words, stem cells regularly utilize a cosuppression-type mechanism to maintain their fate against not only parasite-derived, but also endogenous-borne fate-altering sequences which regularly act to change the fate, i.e., initiate differentiation, of those daughters destined to change fate.

More specifically, the following description of how stem cells and certain of their maturing non-stem cell progeny of higher eukaryotes rear daughters of changed fate as a result of a key fate-altering interallelic communication event that occur at the DNA, RNA, or both levels, wherein the resolution of which triggers a sequence of events leading to an eventual cell fate change in that particular daughter cell. These cell fate mechanisms occur by genetic recombination, changes in epigenetic structure, or RNA degradation, which lead to an expression change or silencing of at least one of the two interacting alleles.

Concomitantly, stem cells and certain of their maturing non-stem cell progeny of higher eukaryotes also rear daughters of unchanged fate, resulting directly from avoidance or inhibition of the interallelic paired event or a fate-altering consequences, for example by a cosuppression-type fate-maintaining protective process against the integrity-threatening paired event.

Strong support for these mechanisms is derived from several factors. First, evidence that a developmentally-regulated allele-specific expression change (i.e. silencing of at least one of the two alleles presumed to interact) does indeed regularly occur at key cell fate transition points, such as, at the stem cell level and beyond, during mammalian development. This is coupled with a demonstration that such allele-specific expression changes do indeed appear to result from actual interallelic communications at the DNA, RNA or both levels.

During certain key cell fate transition points in human development, a noticeable and regular pattern characterized by changes from biallelic to monoallelic expression actives at some well-studied loci (25-30). Both alleles of concern are biallelically expressed in a stem cell or certain of its developing/maturing non-stem cell progeny and, following an allelic interaction by DNA:DNA, RNA:RNA, or RNA:DNA pairing, one of the two interacting homology becomes silenced while the other remains active (i.e. thus monoallelically expressed) in the daughter cell destined to change fate. Also, the daughter of unchanged fate would maintain the state of biallelic expression, for example, due to inhibition of an allelic interaction.

It should be noted that other seemingly less common outcomes of interallelic communications in the daughter of changed fate are also possible, for example, the transition not only from biallelic to monoallelic expression, but also from biallelic to neither allele being expressed, or from neither allele to one or both being expressed.

Three well-studied human loci, in particular (31), follow the general pattern of reduction from biallelic to monoalleic expression during key developmental regulated cell fate transitions. These loci reside within the X-chromosome, certain autosomally imprinted regions, and certain lymphoid-related recombination sites.

With regard to the X-chromosome, certain X-linked genes regularly undergo a change from biallelic to monoallelic expression which follows the change from both X-chromosomes active to one X-chromosome inactivated during the rearing by embryonic stem cells of progeny of changed or differentiated fate (13, 25). That such a reduction to monoallelic expression arises specifically from homologous sequence interactions has also been clearly demonstrated. That is, an untranslated/noncoding RNA species, termed Xist, has been reported which forms a key RNA:DNA complex with homologous X-chromosomal DNA, an event which triggers changes in epigenetic (e.g. methylation, chromatin, nucleosomal) structure leading to silencing of the involved X-chromosome and in turn a cell fate change toward differentiation (13, 25). Interestingly, it has recently been reported that a DNA:DNA pairing event contributes to the perpetuation of such RNA:DNA-initiated fate-altering events (13), thereby indicating that while an homologous RNA:DNA-mediated interaction may initiate a cell fate change, this transition may become more reliably heritable in maturing cells only following subsequent homologous DNA:DNA-mediated events, possibly involving epigenetic exchange, such as, chromatin, methylation, or nucleosomal structure. That RNA-mediated interallelic events may precede or direct subsequent DNA-mediated ones in eukaryotic systems, for example by RNA- or RNA:RNA-mediated DNA methylation (14-16).

Similarly, a host of sequences within certain autosomally imprinted regions, such as, within human chromosomes 11p and 15q, also change from biallelic to monoallelic expression during developmentally regulated cell fate transitions (26-28). Providing additional support of homologous sequence pairing at both RNA and DNA levels.

There is a striking preponderance of untranslated/noncoding overlapping sense and complementary antisense RNA transcripts in these regions strongly indicating that certain RNA:RNA interactions, such as antisense RNA:sense RNA, are active (16, 33). Moreover, like in the X-chromosome case, it has been suggested that such RNA-level inter-homolog communication events and expression changes arising therefrom may similarly precede and possibly direct more heritable DNA-level events, such as changes in epigenetic structure (16).

Indeed, certain of these the imprinted allelic loci have been shown to undergo DNA:DNA association/pairing as normal cellular events (8), as well as exchange of epigenetic structure (e.g. methylation) in certain situations (9, 10).

With regard to certain alluded to lymphoid-related recombination sites, there is also discussed evidence of developmentally regulated fate-altering homologous sequence interactions in a similar manner to those cases. More specifically, similar to the X-chromosome situation, there are data that an untranslated/noncoding RNA species (termed a switch, or S, sequence) associates with homologous chromosomal DNA or forms an RNA:DNA complex and initiates a fate change brought about by subsequent class switch genetic recombination (34-37). Unlike resolution of the X-chromosome-linked RNA:DNA event that is epigenetic in nature and characterized by chromatinization and X-linked silencing, resolution of the lymphoid-related RNA:DNA event, like in the yeast (DNA:DNA) system, involves genetic (DNA:DNA) recombination. Again, a DNA:DNA-mediated event finalizes and perpetuates ordinarily less heritable RNA-initiated fate changes. While the RNA:DNA event preferentially occur in maturing progenitor cells (34-36) that in certain instances occur or originate in renewing stem-like cells (37).

Accordingly, it is conceivable that the lymphoid-related RNA:DNA class switch event as well as other related RNA:DNA events, or the inhibition of such events, e.g. via upstream RNA:RNA interactions, may occur at the level of 1) the stem cell, 2) immediate stem cell progeny (i.e. progenitors), or 3) more maturing progeny. In this way, while the class switch recombination event may normally occur in more maturing progeny, it is also conceivable that such may be ultimately initiated (but kept tonically silenced, e.g. via inhibitory RNA:RNA action) in more immature cells that maintain their fate.

### Prevention of Cell Fate-Altering Interallelic Events by Cells, which Maintain their Fate

As explained, stem cells or certain of their maturing non-stem cell progeny that undergo asymmetric mitotic division concomitantly, in addition to rearing daughters of changed fate, rear daughters of unchanged fate. As will be shown, there is strong evidence that this latter process (i.e. maintenance of a stem cell phenotype, or other maturing non-stem cell phenotype by one of its daughters) is made possible via avoidance or inhibition of the aforementioned cell fate-altering interallelic events or their consequences.

One case in point is the X-chromosome and its expression-related changes enacted during production by embryonic stem cells of differentiated progeny. Embryonic stem cells, upon rearing progeny of changed (i.e. differentiated) fate, concomitantly renew themselves by rearing also daughters of unchanged, i.e. stem cell, fate. During this process, as mentioned previously, production of differentiated progeny is closely associated with a change from biallelic to monoallelic expression of X-linked genes (25, 38).

Continuing, it also appears clear that those daughters of unchanged fate (i.e. stem cell daughters) remain so in large part due to maintenance of biallelic X-linked gene expression. It has recently been reported that an untranslated/noncoding RNA species antisense/complementary to Xist, termed Tsix, inhibits Xist activity (32). That is, Tsix prevents Xist from binding DNA and triggering a fate change by formation of an RNA:RNA, a Tsix:Xist dsRNA inhibitory complex. Tsix may also inhibit Xist action not only via post-transcriptional but also via other, transcriptional, means by promoter blockage or enhancer competition (16). Accordingly, it is predicted that those daughters which express Tsix (or utilize Tsix effectively) maintain their stem cell fate, whereas those daughters which do not effectively utilize Tsix, either by virtue of not expressing Tsix, or by expressing an inhibitor of Tsix, will undergo an uninhibited Xist RNA:DNA-mediated fate change. In this way, the sense RNA Xist changes cell fate toward differentiation, while the antisense RNA Tsix maintains stem cell fate. That a complex series of additional antisense RNA:sense RNA interactions may lie upstream of and modulate Tsix:Xist RNA level interactions is further suggested by reports that other untranslated/noncoding RNA species of overlapping complementarity may lie within this region (39, 40).

In this way, there exists antisense species that are antisense to Tsix (i.e. anti-Tsix) and thus permit unimpeded Xist action, as well as antisense species antisense to this (i.e. anti-anti-Tsix) and thus permit unimpeded Tsix action, and so on (i.e. a long series of overlapping antisenses exists with mutually opposing function). In this way, one attempting to interfere with Tsix action could intentionally block Tsix or more upstream anti-anti-Tsix, etc, whereas one attempting to interfere Xist action could intentionally block Xist or more upstream anti-Tsix.

Of note, a strikingly similar system exists in certain prokaryotes. That is, some coliform bacteria have their fate threatened by the action of parasitic plasmids. More specifically, in this system, a sense RNA binds plasmid DNA (forming an RNA:DNA complex) that primes plasmid replication (41). As a defense against such, an antisense RNA species can bind to the sense RNA thereby preventing it from forming a fate-threatening RNA:DNA complex (41). This system is quite similar to that described above in eukaryotes wherein the antisense Tsix similarly inhibits its complementary sense Xist from forming a fate-threatening RNA:DNA complex. Thus, this general mechanism is highly conserved throughout both eukaryotes and prokaryotes.

The novelty of the present invention is the realization that this mechanism is not only a defense against parasites (e.g. plasmids, viruses, transposons, transgenes, etc) but also a key mechanism in determining cell fate. Thus, manipulation of these mechanisms will affect cell fate (more specifically stem cell fate) and thus has applications in regenerative medicine, cancer.

As noted previously, a non-random preponderance of antisense and sense RNAs resides at certain of the aforementioned autosomally imprinted loci at human chromosomes 11p and 15q—some species of which, like those within X-linked loci, appear to possess overlapping complementarity and thus interact (16, 33). Accordingly, in a similar manner to that described for maintenance of the embryonic stem cell phenotype (i.e. involving antisense RNA-mediated inhibition of a potentially fate-altering sense RNA:DNA-mediated event), so too during developmentally-regulated transitions of other precursor-type cells (of other tissue types, e.g. germline as well as somatic) may certain other antisense RNAs (e.g. transcribed not only from X-regions, but also from other autosomally-located regions, e.g. imprinted) act to modulate or inhibit more downstream fate-altering interallelic (e.g. RNA:DNA or RNA-RNA-mediated) events in daughters of unchanged fate. That is, certain antisense RNAs at imprinted loci may function to prevent the transition from biallelic to monoallelic expression (i.e. maintain biallelic expression) in those daughters of either stem cells or certain of their maturing non-stem cell progeny which do not change fate.

More specifically, as in the X-region, certain imprinted antisense RNAs in a similar manner to Tsix may be expressed in daughters that maintain fate whereas those same antisense RNAs are either not expressed or post-transcriptionally silenced in daughters that change fate—these daughters change fate because a sense RNA in a similar manner to Xist is then free to form fate-altering RNA:RNA or RNA:DNA complexes.

Also, as noted, the lymphoid system utilizes a RNA:DNA event to initiate a key developmentally regulated cell fate change (i.e. class switch recombination). In this case, such a change occur not necessarily at the stem cell level, but rather at the level of maturing non-stem cell progenitors (34-36), although certain stem-like self-renewing cells may undergo a related process (37). Accordingly, it is possible that such a fate change is 1) initiated in a stem cell but not yet realized until a more mature cell is formed, or 2) initiated and realized within a more mature cell. It is also possible, although not yet proven, that an antisense RNA species prevents such an RNA:DNA event (e.g. via inhibition of the RNA species from binding the DNA sequence, like Tsix inhibition of Xist) in cells not undergoing class switch (i.e. those precursor cells, e.g. renewing stem-like cells or lymphoid memory cells, which do not themselves class switch), possibly via tonic antisense:sense-mediated inhibition thereof. Such self-renewing (e.g. stem-like or memory) cells do then yield as their progeny those cells, which can indeed undergo class switch because the antisense RNA is repressed either transcriptionally or post-transcriptionally.

Continuing with the lymphoid system, another cell fate-determining interallelic event, which occurs in a developmentally regulated fashion, is that of VDJ (genetic) recombination (42). In certain immature lymphoid progenitors (some of which may be renewing stem-like cells), a DNA:DNA pairing event resulting in genetic recombination occurs at the VDJ locus (42). Interestingly, this process results in a reduction to monoallelic expression (i.e. allelic exclusion)—similar to the manner in which both X-linked and autosomally imprinted regions undergoing interallelic communications also withstand transitions (from biallelic) to monoallelic expression. While certain lymphoid-related allelic exclusion events (i.e. those involving VDJ recombination) may or may not occur at the level of a renewing stem-like cell (43, 44), other allelic exclusion events at additional loci (e.g. Pax5) do indeed appear to occur specifically in renewing stem-like cells (30, 45). These earlier allelic exclusion events may either causally precede or be independent of those that are recombination-related. Accordingly, lymphoid-related transitions to monoallelic states, some of which may arise directly from interallelic communications, may 1) be initiated and concluded at the stem cell stage, 2) be initiated at the stem cell stage but concluded in maturing non-stem cells, or 3) be initiated and concluded in maturing non-stem cells.

A question then arises as to whether the allelic exclusion events (e.g. at the VDJ or Pax5 loci) are preceded or modulated by RNA level events, as appears to be the case at X-linked and at autosomally imprinted loci. The evidence is highly suggestive that, as predicted, such may indeed occur—i.e. that the initial choice of which allele will undergo VDJ recombination actually occurs earlier than the recombination event itself, a choice possibly ultimately arising from early RNA-mediated events, only later to be fully carried out more permanently by the DNA:DNA event—an emerging theme. For instance, it has been demonstrated that certain epigenetic (e.g. demethylation) events precede and direct the choice of which allele will eventually undergo recombination (46). Considering evidence that epigenetic (methylation-related) events at X-linked and autosomally imprinted loci appear indeed to be preceded by RNA-level events, coupled with increasing evidence that an array of epigenetic (methylation-related) processes in a variety of organisms appear also to be ultimately RNA-initiated (16, 47), the following novel scenario is proposed: a specific antisense RNA acts in renewing lymphoid cells to prevent the fate-altering action of a sense RNA—the antisense RNA does not act in daughters of changed fate (i.e. daughters undergoing differentiation) because such daughters either 1) do not express the antisense, or 2) post-transcriptionally inhibit the antisense RNA. The sense RNA, uninhibited in daughters of changed fate, is thus free to form a sense RNA:DNA complex on one particular allele. Such, in turn, leads to epigenetic (methylation) alterations thereby leaving a differentiating mark on one allele versus the other which will in turn lead to one allele being recombined via VDJ recombination and the other not (i.e. allelic exclusion).

In this way, renewing stem-like cells of lymphoid, or other tissue type, origin or certain of their maturing non-stem cell progeny can be manipulated for the study of regenerative medicine or cancer or the treatment thereof by inhibiting a fate-maintaining antisense RNA or a fate-changing sense RNA, or vice versa—i.e. a fate-changing antisense RNA or a fate-maintaining sense RNA.

### Additional Upstream RNA:RNA Events that May Control the Antisense:Sense Events

Of note, each of the 3 highlighted loci (i.e. X-linked, autosomally imprinted, and lymphoid-related) harbor enhancer regions that appear to play a role in allelic exclusion decision-making (48-50). This is key because, as will be explained, it appears that RNA:RNA events acting at enhancer regions may lie upstream of and thus modulate the more downstream fate-determining antisense:sense interactions discussed in the last section. More specifically, it has been suggested that certain upstream enhancer regions themselves may undergo allelic exclusion-type events (51)—an assertion consistent with recent findings that allelic exclusion in the form of imprints of certain loci appears indeed to derive initially from enhancer regions which themselves becomes allelically excluded (i.e. imprinted) first (48). Moreover, there is increasing evidence that allelic exclusion choice at enhancer regions is directed by RNA-level interallelic actions, e.g.: 1) some enhancer regions (e.g. at X-chromosomal or lymphoid-related sites) harbor antisense-type sequences (49), and 2) certain lymphoid-related enhancer regions encode short RNAs (e.g. kappa NE, a 27 bp enhancer sequence with homology to certain repetitive sequences), whose action is abolished by another short RNA species (i.e. kappa BS, a 30 bp sequence) suggesting possible RNA-level interactions (50). That enhancer region modifications may then affect more downstream allelic (e.g. DNA:DNA) interactions has also been suggested (51). Accordingly, upstream of the aforementioned fate-determining antisense:sense RNA interactions may lie a controlling upstream enhancer region the allelic exclusion of which in part directs the downstream fate-determining events (e.g. via modulation/inhibition of downstream RNA:DNA or RNA:RNA-mediated cell fate-alterations), which may include 1) choice of which allele is ultimately excluded in the daughter of changed fate, and 2) the inhibition of which maintains fate in the daughter of unchanged fate.

Such enhancer-related RNA events may lie downstream still of other more upstream RNA-related events the nature of which may ultimately originate allelic exclusion choice. Case in point, it has been shown in fission yeast that asymmetry (i.e. an interallelic communication event occurring in one daughter but not the other) arises directly from the asymmetry of DNA strand replication (5). That is, yeast daughters of unequal fate are produced because one daughter of unchanged fate receives the leading DNA strand, while the other daughter destined to change fate receives the lagging DNA strand. The lagging strand in this case is not completely “healed” in that there is a mark/imprint/memory of its past which takes the form of an RNA moiety within the Okazaki fragment that has not yet been fully converted to DNA (5). This lagging strand mark then promotes a subsequent interallelic (DNA:DNA) event that results in a fate change (via recombination)—possibly related to the manner by which other type gaps can promote recombination/repair (52, 53).

Considering that such processes are well conserved, the idea that mammalian stem cells also differentially segregate DNA strands (as previously suggested by Cairns (54), and others since) appears quite sound. More specifically, evidence indicates that lagging strands may be preferentially segregated to non-stem cell daughters of changed fate while leading strands are maintaining within stem cell daughters of unchanged fate (54). Accordingly, the following novel proposal is made: lagging strands within non-stem cell daughters promote exchange like in yeast but of epigenetic rather than genetic or recombination material—an exchange which in turn triggers a cell fate change. Stem cell daughters of unchanged fate maintain their fate because of maintenance of leading rather than lagging strands.

With regard to the lagging strand-initiated epigenetic fate-altering event, there is considerable evidence that such may be directed by the action of a reverse-transcribed entity (e.g. retroelement, or otherwise). For example, reverse transcription activity has been shown to be present (e.g. via recombination or repair) at the MAT site in certain yeast, albeit such constitutes a rare phenomenon but indicative of a related more common process (55). Considering that retroelement activity may be increased by the presence of gaps (e.g. as are seen in lagging strands) (52, 53), the following novel idea is proposed: lagging strand gaps cause reverse transcription activity (e.g. of retroelements or other entity) that in turn leads to inter-allelic exchange and fate alterations. For example, reverse transcription-mediated inter-allelic strand invasion can promote exchanges of epigenetic or genetic material (52, 53), and a related process has been implicated in certain (lymphoid-related) cell fate transitions (56). Since reverse transcription is often primed by a tRNA species (57), certain tRNA species may act as fate-altering sense RNAs which change fate by initiating reverse transcription of entities resulting in inter-allelic (DNA:DNA) communication events and fate changes. In this way, there also exists antisense RNA species which block tRNA action in this manner in non-stem cell daughters such that only one of 2 alleles undergoes an expression change, thereby maintaining the biallelic to monoallelic expression transition rather than a biallelic to no allelic expression transition. Candidate antisense species of these types are snoRNA-types. This is important for ensuring there is no toxicity to normal stem cells when devising anti-stemline therapy for cancer.

More specifically, therapies designed to inhibit fate-maintaining antisense RNAs (e.g. snoRNA-type) will lead to a tRNA mediated fate change (i.e. loss of immortality/eradication) of cancer stemline cells since cancer stemline cells, by virtue of dividing symmetrically, randomly segregate leading/lagging strands. On the other hand, since normal stem cells regularly segregate out lagging strands to non-stem cell daughters, therapies designed to affect fate-maintaining snoRNA-types will not affect normal stem cells that will thus remain unharmed.

Interestingly, L1 retroelements have recently been implicated in some phases of cell fate changes occurring during X-chromosome inactivation (58). One scenario is that such tRNA-primed retroelement activities cause other fate-altering sense RNAs to be expressed or bind DNA (thereby triggering a cell fate change). If so, might such retroelements be inhibited from acting in daughters that maintain their fate? Or more specifically, might regular inhibition of the action of such elements occur at the level of inhibition of its tRNA primer to maintain the immortal fate of stem-like cells, e.g. within a cancer stemline? Might such inhibitory activity also, or instead, occur not only in the daughter of maintained fate but also in the allele that remains active after a biallelic to monoallelic expression transition in the daughter that changes fate? In this way, blockage of such inhibitory activity would cause a stem-like cell (e.g. within a cancer stemline) to become mortal/eradicated.

Interestingly, a related process exists in certain bacteria. That is, certain fate-altering parasitic elements (i.e. plasmids) use an RNA-based primer to replicate themselves—a primer which can be inhibited by a native antisense RNA species (59, 60). But even more interestingly, is the demonstration that the primer RNA species (which is inhibited by the antisense RNA) shares homology with certain tRNAs (59, 60)—thereby indicating that a tRNA-like species priming an fate-altering entity (e.g. prokaryotic plasmid, or possibly eukaryotic retroelement) can indeed be inhibited by an endogenous antisense RNA. Considering that some of such bacterial RNAs share homology with human mitochondrial met tRNA (60), the following novel concept is proposed: eukaryotic cells harbor endogenous antisense RNAs which are complementary to and block the tRNA species which prime potentially fate-altering retroelement activities. Of note, there are indeed a number of snoRNA species with no clear RNA substrate (61-63), suggesting that such snoRNAs may bind other RNA types (e.g. tRNAs) in an antisense manner (e.g. to maintain cell fate).

Recently, certain snoRNA species have been shown to be imprinted (64)—findings consistent with a scenario wherein such imprinted snoRNAs undergo a developmentally regulated biallelic to monoallelic expression change during the production by stem cells of daughters of changed fate. In this way, programmed decreased snoRNA expression (from one allele, i.e. following the biallelic to monoallelic change) should then allow its cognate tRNA to prime a retroelement that in turn triggers a fate change. In this case, the tRNA species may comprise a family of fate-altering sense RNAs, and the snoRNA species may comprise a family of fate-maintaining antisense RNAs.

Interestingly, the tRNA-type and snoRNA-type species appear to harbor key functional domains in the range of 10-25 bp (a size range which, as will be discussed later, has additional relevance with regard to RNA-interference-type processes). More specifically, tRNA primer sites have been reported in the 18 bp range (65), and rRNA binding sites for corresponding snoRNA have been reported in the 10-21 bp range (66, 66A). Also, Okazaki intermediates containing RNA species have been reported in this size range (67, 67A), and interestingly certain antisense species have also been detected at Okazaki sites (68)—indicating possible RNA:RNA modulation.

Also of note, it has recently been reported that an untranslated RNA species complexed with a protein species in the form of a riboprotein termed MRP is associated with certain cancers such as lymphomas (69, 70). Even more interesting is the finding that this species functions in both mitochondrial and nucleolar biology (69, 70). Accordingly, this molecule or a related species acts within a program wherein mitochondrial tRNA-like species function as a fate-altering sense RNA, and snoRNA-type molecules function as fate-maintaining antisense RNA.

In this way, the MRP riboprotein may act or share homology with other riboproteins or RNA species that function in cell fate decisions via the hypothesized tRNA (possibly mitochondrial, or nuclear, in origin)-snoRNA (possibly nuclear in origin) axis. Also of note is the reported role of MRP in differential DNA strand synthesis (69, 70), wholly consistent with the proposed idea concerning lagging strands and fate-determination.

### General Model of Fate-Altering RNA-Mediated Events and Modulation/Control Thereof

Based on data cited in the previous sections, the following mechanism is advanced: A series of upstream RNA:RNA interactions (i.e. antisense RNA:sense RNA in nature) with overlapping complementarity modulate/inhibit other downstream antisense RNA:sense RNA actions which ultimately affect a key downstream potentially cell fate-altering interallelic communication event (RNA:DNA, RNA:RNA, or possibly DNA:DNA in nature). Amid or upstream of these interactions lie a contributing role for enhancer-related sequences in affecting the outcomes of such, e.g. that themselves withstand RNA:RNA-initiated decisions such as allelic exclusion events that then get perpetuated. Also, upstream still of such enhancer-related events may lie lagging strand-initiated reverse transcription activity (e.g. possibly primed by tRNA-type species) that act to change fate. In this way, certain antisense species (e.g. snoRNA-type) may maintain one allele in its prior state, while the uninhibited tRNA-type species can alter expression of the remaining allele in the daughter that will change fate. Since the tRNA-type activities emanate from the lagging strands which are present in cancer stemlines but not in normal stem cells which asymmetrically segregate out lagging strands thereby maintaining leading strands, therapeutic inhibition of antisense (snoRNA-type) activities that result in uninhibited tRNA-type (mortality-causing) activity only in cancer stemline cells while sparing normal stem cells of toxicity.

In this way, during cell fate transitions at the level of stem cells or possibly certain of their maturing non-stem cell progeny, those daughters of changed fate undergo the fate-altering interallelic event (either because an inhibitor of such, e.g. an antisense RNA, is either not expressed, or is expressed but is itself blocked, e.g. post-transcriptionally). On the other hand, those daughters of unchanged fate inhibit the fate-altering interallelic event, e.g. via expression of an inhibitor of such (e.g. via utilization of an antisense RNA which blocks fate-altering RNA:DNA- or RNA:RNA-mediated events) or via blockage of another inhibitor of the antisense (i.e. an antisense to the antisense).

Accordingly, it is proposed that stem cells and certain of their non-stem cell progeny rear 1) progeny which change fate because such express a sense cell-fate-determining untranslated/noncoding RNA (sense-cuR)—a species which acts via RNA:DNA or RNA:RNA mediated events to bring about a cell fate change, and 2) progeny which maintain fate because such express an antisense cell-fate-determining untranslated/noncoding RNA (anti-cuR) which is complementary to and therefore inhibitory of the potentially fate-altering sense-cuR. Additionally, there is not just one anti-cuR or sense-cuR species but rather a series of such—e.g. an anti-anti-cuR, an anti-anti-anti-cuR. As will be shown, this series may initiate and conclude in stem cells, initiate in stem cell and conclude in maturing non-stem cell progeny, or initiate and conclude in maturing non-stem cell progeny.

More specifically, while some of the aforementioned interallelic events clearly occur at or close to the level of the stem cell (e.g. X-linked exclusion), others may occur at fate transitions of maturing progeny of stem cells (e.g. certain autosomally imprinted or lymphoid-related exclusion events). That is, in a similar manner to which maturing non-stem cell progeny may inherit certain features from their stem cell mother (e.g. some surface antigens), so too do they inherit other features including the mechanisms involving fate-determining interallelic communications and resolutions thereof. Thus, it is possible that: 1) certain interallelic communication events and their resolution occur solely in stem cells (e.g. X-linked exclusion), 2) certain interallelic communication events and their resolution occur in non-stem cells (i.e. maturing progeny of stem cells, having inherited and exploited such a mechanism from their stem cell mother) (e.g. exclusion of certain imprinted loci might at times occur subsequent to the stem cell stage), or 3) certain interallelic communication events initiate in stem cells but are resolved in more mature progeny of stem cells—e.g. certain exclusion events during lymphoid development (e.g. or RNA-initiated epigenetic changes) do appear to occur at or near the stem cell level (46), while others (e.g. recombination of immunoglobulin loci) may or may not (43, 44)—whether causally linked or mutually independent remains to be seen.

In addition to those 3 human loci highlighted, numerous other regions have been found to possess certain allelic differences (e.g. in methylation pattern)—findings that strongly suggest that meaningful homologous communications may be both a regular finding in developing (e.g. stem and maturing) cells as well as a process that involves multiple allelic sites (31). Accordingly, the untranslated/noncoding RNA species which are fate-altering and fate-maintaining are plentiful with sequence specificity particular to cell maturation stage (i.e. stem cell versus maturing cell), communicating allelic sequences involved, cell type, tissue type.

Thus, procurement of these RNA species into two groups (i.e. fate-altering and fate-maintaining) will allow one to evaluate at what maturation stage, what allele, and what cell type such act—and accordingly, which are the most appropriate targets for which to design therapeutics seeking to manipulate cell fate decisions at the level of stem cells and certain of their maturing non-stem cell progeny for the purposes of studying or treating conditions relating to regenerative biology/medicine or cancer. It should also be noted that certain of the aforementioned RNA species might be complexed with protein, and function as a riboprotein (e.g. MRP, as mentioned) (69, 70).

### DETAILED DESCRIPTION OF THE INVENTION

The present invention relates to a method of manipulating a cell fate-determining mechanism which includes the manipulation of cells, stem cells, and certain stem cell progeny. The present invention can be utilized to manipulate cells for purposes of studying or treating conditions related to developmental biology, regenerative biology/medicine, cancer, aging, as well as other conditions.

In general, the invention, is directed to a method of manipulating the fate of a cell. This is accomplished by contacting the cell with a compound that affects a cell fate-determining untranslated/noncoding RNA species (cuR), which results in the cell changing or maintaining cell-fate.

Furthermore, the fate of the cell can also be manipulated by contacting the cell with a cuR or a compound that modifies a cuR or a protein/riboprotein that is associated with a cuR, which also results in the cell changing or maintaining cell-fate. The present invention further encompasses the use of compounds that mimic cuRs.

Another feature of the invention is a therapeutic method of treating a condition in a subject by manipulating a fate-determining mechanism in a cell by administering to the subject an effective amount of a compound. The compound administered inhibits, maintains or activates the fate-determining mechanism by affecting a cell fate-determining untranslated RNA species (cuR), a cuR-binding DNA sequence, a modified cuR, a cuR-associated protein, or a cuR-riboprotein.

It is preferable that the subject is an animal and more preferable that the subject is a human.

The invention further encompasses a method of procuring cuR from a cell. Preferably from a stem cell or cancer cell. The method involves use of standard techniques of nucleic acid isolation and purification.

In addition, the invention provides a method of identifying compounds that interacts with a cuR. This is accomplished by contacting an identified cuR with a biological or chemical library and identifying those compounds that interact with the cuR.

A preferred embodiment of the invention relates to a method of treating cancer, wherein cancer stem cells (largely slow-growing) and progeny cells (largely faster-growing) can be specifically targeted based on a fate-determining mechanism. This method encompasses the manipulation of the fate-determining mechanism in both slow-growing and fast growing cancer cells.

The invention also encompasses a method of manipulating the fate-determining mechanisms to expand and contract cell pools for purposes of regenerative medicine.

More specifically, the invention relates to use of the native fate-determining cosuppression, RNA-interference or RNA-editing mechanisms to manipulate the fate of cells for the purpose of studying or treating conditions relating to regenerative biology, cancer, developmental biology and aging.

The invention provides methods of using cosuppression. RNA-interference and RNA-editing mechanisms naturally used by long-lived cells, such as stem cells, to protect their cell-fate against sequences would alter cell-fate. These naturally occurring protection mechanisms against endogenous sequences are used by cells, including stem cells, to ensure that the cell-fate is not changed during normal development. In this way, these cells utilize this fate-maintaining mechanism to renew themselves during normal development. Cancer stem cells also utilize this mechanism to renew themselves during tumorigenesis. Accordingly, the invention provides methods of manipulating these mechanisms so as to alter cell fate decisions in normal or neoplastic cells.

The fate-determining mechanism involving cosuppression, RNA-interference, and RNA-editing processes involves the formation of paired nucleic acid structures which includes RNA:RNA, RNA:DNA, DNA:DNA as well as other related complexes which if left alone can at times change fate and at times maintain fate. If the nucleic acid complexes are not left alone, but are resolved then cell-fate will be changed or maintained. Examples of molecules that resolve these nucleic acid complexes include nucleic acids, proteins, riboproteins, or other molecules, which degrade or in some way modify such paired structures.

A specific example is a stem cell that rears daughters of changed fate when such fate-altering sequences are unimpeded, but rear daughters of unchanged fate when such fate-altering sequences are impeded. Such sequences are impeded by a cosuppression, RNA-interference-type, and RNA-editing-type mechanism wherein a paired nucleic acid structures, such as, dsRNA or RNA:DNA, are degraded or modified by a program of molecules, which include nucleic acids, proteins, riboproteins and other and thereby maintaining fate. Maintenance of fate can either occur by expression of an antisense RNA that forms a paired nucleic acid structure with its complement sense RNA or expression of a program of molecules that include nucleic acids, proteins, riboproteins and other, which degrade or modify paired nucleic acid structures. These sequences are unimpeded and thus cause a fate change when the inhibitory antisense RNA is either not expressed or is post-transcriptionally repressed, or the program that degrades or modifies paired nucleic acid structures is not expressed. There are certain key antisense RNAs that maintain fate. Thus if the antisense RNA is inhibited the cell-fate will be changed.

In addition there are certain key sense RNAs that change fate. Therefore, by inhibiting the sense RNAs the cell will maintain fate.

It is important to note that the fate-determining mechanism and program includes sense and antisense RNA, other nucleic acids including DNA, proteins, riboproteins, and other molecules.

Such fate-determining sense and antisense RNAs and other nucleic acid structures that will or are paired can be procured from cells changing or maintaining fate and then subsequently cloned and identified by sequencing. Reagents/drugs can then be developed that interfere with specific sense and antisense RNAs for the purpose of manipulating the fate of cells for the study or treatment of conditions relating to regenerative biology/medicine, cancer, and other. Such reagents/drugs affect RNA targets so can be ribozymes, antisense, double-stranded RNA, peptide nucleic acids, or other RNA-affecting/binding species which can be designed given a target sequence of interest. Also, assays can be readily developed and small or large molecule libraries readily screened (in a high throughput manner) to identify those compounds, which best affect/bind RNA targets of interest. Also, assays can be readily developed and small or large molecule libraries readily screened (in a high throughput manner) to identify those compounds, which best affect/bind those targets of interest that are nucleic acids, proteins, riboproteins, and other molecules.

Additionally, the present invention exploits certain novel aspects of stem cell biology to carry applications not only with regard to the study and treatment of cancer, but also the study and treatment of other conditions relating to stem cell and developmental biology such as regenerative biology/medicine. Regenerative medicine, an alternative or adjunct to transplantation medicine, involves the repair/renewal of tissues of plants or animals where the tissue is either damaged or in need of some functional improvement.

Accordingly, it should be noted that while applications relating to cancer seek to diminish the proliferative potential of cells, stem cells, or certain faster-growing progeny, applications relating to regenerative biology first seek to expand the pool of cells via enhancing their proliferation, and then contract the cell pool through directing differentiation toward particular cell types. Additionally, while these tactics do work at the level of the stem cell, since the mechanisms to be described are also passed on by stem cells to their non-stem cell maturing progeny, such applications affect in an intentional or unintentional manner also those more mature non-stem cells in cancers and regenerating tissues.

Efforts that affect fast-growing or non-stem-like cells are within the scope of this invention. More specifically, efforts that target such fast-growing mortal cells by targeting the cell fate-determining mechanisms described are considered part of this invention—since such mechanisms are clearly inherited by such fast-growing mortal non-stem cells from their immortal stem-like precursor cells.

In general, the present invention relates to a method of screening for any type of molecule involved in the fate-determining mechanism. This may include RNA, DNA, proteins, riboproteins and other fate-determining molecules.

In addition, the invention also includes general methods of designing or screening for a compound that interacts with any of the identified fate-determining molecules. The invention further encompasses those compounds found to manipulate a fate-determining mechanism.

Again, the novelty of the present invention is the use of a mechanism normally thought to function as a defense against parasitic elements (e.g. plasmids, viruses, transposons) in a new way—that is the use and manipulation thereof of this mechanism that acts also in cell fate-determination to affect cell fate decisions for the purpose of studying or treating conditions relating to cell fate decisions such as regenerative biology/medicine, cancer, aging. This mechanism is known by many names including cosuppression, RNA-interference, quelling, post-transcriptional gene silencing, as well as a related paired nucleic acid structure-related process called RNA editing which modifies double-stranded RNA. The novel use of this mechanism characterized by the formation of paired nucleic structures and at times their resolution via degradation/modification by a program of molecules (nucleic acids, proteins, riboproteins, and other) derives from the realization that this mechanism acts not only as a defense against parasitic elements but is also key in defending cells against other challenges to their fate (e.g. differentiation-induction) as normally occurs during development.

This mechanism is used by stem cells to maintain their fate to reliably renew themselves during development, as well as by certain non-stem cell progeny of stem cells that have inherited the mechanism from them. In this way, manipulations of this mechanism can be used for regenerative biology/medicine purposes, such as to expand or contract stem cell pools, or cancer to contract/eradicate a cancer stemline. The mechanism also includes those modifications of paired nucleic structures including double-stranded RNAs (e.g. RNA-interference or RNA-editing) used not only by cells as an anti-viral defense, but also in a novel way by stem-like cells during development to maintain their fate.

Thus, the invention includes methods of manipulating the mechanism thereof so as to effect cell fate, more specifically stem cell fate, and thus has applications in regenerative medicine, cancer. The fate-determining mechanism is made up of nucleic acids, protein, riboprotein, and other components. Thus any or all of these may serve as targets and are encompassed by the invention. The invention embodies methods of treating cancer by manipulation of the fate-determining mechanism. In addition the invention encompasses a method of targeting cancer cells based on a specific fate-determining mechanism. By targeting the fate-determining mechanism, differentiating between normal cells and cancer cells is enabled, thus allowing for cell specific treatments, which could be beneficial to the treated subject and reduce damage to normal cells.

t-RNA-like means a molecule that has the same sequence and structure as a tRNA but a different function.

snoRNA-like means a molecule that has the same sequence and structure as a snoRNA but has a different function.

In general, the fate of a cell can be changed or modified by contacting the cell with a compound that affects cuR, which results in the cell changing or maintaining cell-fate. The cell is preferably a stem cell, stem cell progeny, cancer stem cell or cancer stem cell progeny and more preferably a stem cell or cancer cell.

A cuR is a cell fate-determining untranslated RNA species. A cuR may be genomically derived, organelle derived, tRNA's, snoRNA's, lagging strand-derived, okazaki fragment-derived, or derived from some other nucleic acid-harboring entity, as well as be transposon-derived, such as, a transposon or retroelement sequence or primer, or other parasitic-type element, such as, satellite sequences, repeat sequences, endogenous viral sequences, aberrant sequences.

A cuR can be a sense-cuR which binds DNA to affect cell fate by changing or maintaining fate. The DNA that binds a cuR is a cuR-binding DNA sequence, usually. A cuR can be an anti-cuR which binds RNA to affect cell fate. A cuR can affect cell fate alone or in conjunction with other cuR's, cuR-associated proteins, or as part of a protein complex such as a cuR-riboprotein. The cuR often located in the nucleus or cytoplasm of the cell.

An additional feature of the invention is a method of manipulating the fate of a cell by contacting the cell with a cuR or a compound that modifies a cuR or a protein/riboprotein that is associated with a cuR, which also results in the cell changing or maintaining cell-fate.

Modified cuR's are cuR's that either alone, such as tRNA's, snoRNA's, or aberrant RNA's or the formation of RNA:DNA, double stranded RNA's, such as an anti-cuR:sense-cuR complex are unmodified but inhibited from acting in some manner. This can occur, for example, by small temporal RNA inhibition of translation of its complement by binding to its untranslated region (UTR), or by modification conducted by RNA-mediated DNA methylation or chromatin changes, RNA-silencing, RNA-interference, RNA-editing, snoRNA-mediated modifications, or other related processes that lead to the production of DNA methylation or chromatin changes, small interfering RNA's, small temporal RNA's, microRNA's, aberrant RNA's, snoRNA-modified RNA's, edited RNA's, or other modified RNA species.

The compound used in the methods of the invention can be an molecule affecting the cell-fate mechanism, but preferable is an RNA, DNA, double-stranded RNA, a ribozyme, a protein, a riboprotein or a vaccine, or a small molecule.

The invention further encompasses a method of manipulating the fate of a cell by contacting the cell with a compound that modifies a cuR, a protein or riboprotein associated with a cuR, or by simply mimicking a cuR, which results in the cell changing or maintaining cell-fate.

Another feature of the invention is a therapeutic method of treating a condition in a subject by manipulating a fate-determining mechanism in a cell by administering to the subject an effective amount of a compound. The compound administered inhibits, maintains or activates the fate-determining mechanism by affecting a cell fate-determining untranslated RNA species (cuR), a cuR-binding DNA sequence, a modified cuR, a cuR-associated protein, or a cuR-riboprotein.

It is preferable that the subject is an animal and more preferable that the subject is a human. These therapeutic methods are not toxic to humans because normal stem cells of a given tissue type are more numerous than cancer stem cells of that tissue such that a dosing regimen can be determined wherein a clinically relevant number of cancer stem cells can be eradicated while sparing a clinically relevant number of normal stem cells.

It should be appreciated that even if normal tissues were damaged, regenerative medicine and more standard transplantation maneuvers can be employed to repair/replace damaged tissues. Also, many common cancers (e.g. breast, prostate, colon) derive from expendable tissues which would not need to be repaired or transplanted. Also, since cancer stem cells divide symmetrically (i.e. maintain both leading and lagging DNA strands) while normal stem cells divide asymmetrically (i.e. maintain solely leading strands), cuR's created by lagging strand-initiated transcription will serve as targets of cancer stem cells and not normal stem cells.

The invention further encompasses a method of procuring cuR from a cell, preferably from a stem cell or cancer cell. The method involves use of standard techniques of nucleic acid isolation and purification.

In addition, the invention provides a method of identifying compounds that interacts with a cuR. This is accomplished by contacting an identified cuR with a biological or chemical library and identifying those compounds that interact with the cuR.

A preferred embodiment of the invention relates to a method of treating cancer, wherein cancer stem cells (largely slow-growing) and progeny cells (largely faster-growing) can be specifically targeted based on a fate-determining mechanism. This method encompasses the manipulation of the fate-determining mechanism in both slow-growing and fast growing cancer cells.

The invention also encompasses a method of manipulating the fate-determining mechanisms to expand and contract cell pools for purposes of regenerative medicine.

More specifically, the invention relates to use of the native fate-determining cosuppression, RNA-interference or RNA-editing mechanisms to manipulate the fate of cells for the purpose of studying or treating conditions relating to regenerative biology, cancer, developmental biology and aging.

The mechanism involving RNA-silencing, RNA-interference, RNA-editing, snoRNA-mediated modifications, tRNA-primed events, and other homologous nucleic acid interactions of RNA:RNA, RNA:DNA, DNA:DNA and their resolution may act not only as a cell fate determining mechanism but also as a protective mechanism for stem cells and non-stem cells against parasitic elements such as viruses, transposons. Accordingly, the application teaches that activation or inhibition of this mechanism will enhance or eradicate a stem cell, cancer stem cell, or cancer cell due to the inhibition or derepression of damage-inducing parasitic elements in the cell.

### A Novel Mechanism of Cell Fate-Determination

The present application utilizes the unique teachings that stem-like cells and certain of their maturing non-stem cell progeny utilize a primitive defense mechanism (well-described in the literature, but in other contexts) to maintain fate. The defense mechanism has been described in cells that protect their fate from parasitic-like elements (e.g. viruses, transposons), but never before in stem-like cells as a regular part of their life cycle wherein endogenous (non-parasitic) differentiation-inducing sequences are managed. The fate-maintaining mechanism in this case involves 1) the recognition of potentially fate-threatening untranslated/noncoding RNA species (i.e. sense-cuRs) that if left uninhibited will form RNA:RNA or RNA:DNA complexes that change cell fate (e.g. as normally occurs in daughter cells that change fate/differentiate), 2) the blockage of such species, by cells that maintain their fate, via use of a protective antisense species (i.e. an anti-cuR) which forms a dsRNA structure (or other RNA- or DNA-level inhibitory complex with its sense-cuR complement, e.g. involving DNA level enhancer/promoter competition/blockage), and 3) modification or degradation of the dsRNA structure, if/when formed, thereby preventing the sense-cuR from acting (i.e. changing cell fate) by a program of nucleic acids, proteins, riboproteins, or other molecules which may include RdRp-like (e.g. qde1, sgs2, ego1), eIF2C-like (e.g. qde2, rde1, RecQ-like (e.g. qde3), RNase D-like (e.g. mut-7), (135), piwi, hiwi, and RMRP (the RNA component of the riboprotein RNase MRP), as well as RNA-editing-related proteins such as adenosine deaminases (e.g. ADAR).

This protective mechanism acts preferentially in cells that maintain their fate versus change their fate because either 1) anti-cuRs are differentially expressed in cells that maintain their fate (and either not expressed or post-transcriptionally repressed in cells that change their fate), or 2) anti-cuRs are equally expressed in both cells that maintain and change their fate, but the mechanism itself (which includes nucleic acids, proteins, riboproteins, and other molecules) which resolves dsRNAs is preferentially active only in those cells that maintain their fate. In other words, in this case wherein cells both of maintained and changed fate harbor anti-cuR:sense-cuR complexes, only the former (cells of maintained fate) express the mechanism which acts on such (i.e. dsRNA degradation/inhibition).

Accordingly, the aforementioned protective fate-maintaining mechanism can thus serve as the basis for high throughput reagent/drug screens to identify those reagents/drugs that best interfere with the mechanism (or certain of its components, e.g.: anti-cuRs or sense-cuRs). Such reagents/drugs identified by the screen can then be used for the study or treatment of conditions relating to cell fate issues such as regenerative biology/medicine or cancer. The novel use of the mechanism involving the untranslated/noncoding RNA function, such as, RNA:RNA and RNA:DNA, and resolution thereof to affect cell fate. This mechanism may include RNA, DNA, proteins, riboproteins and other fate-determining molecules. These mechanism and fate determining molecules can serve as a target and basis for a reagent/drug screen to study or treat conditions relating to regenerative biology/medicine, cancer was not previously disclosed by U.S. Pat. No. 6,004,528 which itself teaches other methods of eradicating a cancer stemline. Reagents/drugs that inhibit fate changes will be used to expand cells (e.g. stem-like pools, as for regenerative purposes) as will be described, whereas reagents/drugs that cause fate changes will be used to limit cells (e.g. stem cell pools, as for anti-cancer stemline purposes). These concepts can likewise be applied to all fate-determining molecules whether a nucleic acid, a protein, a riboproteins, other fate-determining molecule or combinations of molecules.

### Manipulations of the Novel Untranslated/Noncoding RNA-Based Mechanisms

As stated, it is proposed that a collection/series of cell fate-determining untranslated/noncoding RNA species (cuRs) is composed of 2 subgroups: 1) those species that maintain cell fate (i.e. anti-cuRs), and 2) those species that change cell fate (i.e. sense-cuRs). In actuality a more complex situation exists wherein a series of cuRs, with overlapping complementarity, function to inhibit one another, e.g.:
sense RNA changes fate (a.k.a. sense-cuR)
antisense RNA (blocks the sense RNA) maintains fate (a.k.a. anti-cuR)
anti-antisense RNA (blocks the antisense RNA, thus functionally equivalent to sense RNA) changes fate (a.k.a. sense-cuR)
anti-anti-antisense RNA (blocks the anti-antisense RNA, thus functionally equivalent to antisense RNA) maintains fate (a.k.a. anti-cuR)
anti-anti-anti-antisense RNA (blocks the anti-anti-antisense RNA, thus functionally equivalent to anti-antisense RNA) changes fate (a.k.a. sense-cuR)
and so on, wherein an even number of anti's denotes changers of fate (i.e. sense-cuR-like), and an odd number of anti's denotes maintainers of fate (i.e. anti-cuR-like)

Accordingly, for the mere sake of simplicity, the term “sense-cuR” will include all cuR species which change cell fate, e.g. in addition to sense-cuR, also those species which inhibit the effects of anti-cuRs such as anti-anti-cuR, anti-anti-anti-anti-cuR (i.e. an even number of anti's). Similarly, the term “anti-cuR” will include all cuR species which maintain cell fate, e.g. in addition to anti-cuR, also those species which inhibit the effects of sense-cuRs such as anti-anti-anti-cuR, (i.e. an odd number of anti's).

Accordingly, manipulation of the cuRs, e.g. via use of reagents/drugs that interfere with/inhibit the action of such species (i.e. reagents/drugs specifically able to bind/affect RNA-based cuR targets such as ribozymes (70), antisense (70), double-stranded RNA (dsRNA) (12, 17), antibodies to RNA or dsRNA (71), protein nucleic acids (PNA's) (72), small molecules that affect/bind RNA (73-76), or other RNA-affecting/binding molecule), will necessarily affect (i.e. change or maintain) cell fate. Mote specifically, use of the reagents/drugs to inhibit an anti-cuR and its fate-maintaining function is expected to do the opposite (i.e. change cell fate), while use of the reagents/drugs to inhibit a sense-cuR and its fate-changing function is expected also to do the opposite (i.e. maintain cell fate). Because of such, and according to the present invention, the cuRs will clearly serve as key targets for the readily enabled development of novel reagents/drugs that will necessarily by virtue of affecting/binding cuR targets affect (i.e. maintain or change) cell fate. Such reagents/drugs will be invaluable for the experimental study or clinical treatment of conditions relating to cell fate choices which includes, but is not exclusive to, regenerative biology/medicine, cancer, aging.

With regard to applications in the regenerative biology/medicine space, the present invention teaches how the cuR targets can be readily procured (by methods which will be discussed) and reagents/drugs readily developed therefrom (e.g. via intelligent design or reagent-drug library screens) for the purpose of manipulating the fate of stem cells or possibly certain of their maturing non-stem cell progeny in vitro, ex vivo, or in vivo for experimental study or clinical use. Considering that current strategies in regenerative biology/medicine concern both expanding stem-like cells as well as causing stem-like cells to differentiate toward a particular tissue type (i.e. that tissue type requiring regeneration/repair) (77), the present invention will indeed enable such but by the development of new and unique reagents/drugs, which will in a completely novel way, increase the number of precursor cells (i.e. expand the stem cell pool) but will do so specifically by maintenance of (a stem cell) fate by inhibiting fate-altering sense-cuRs, as well as direct differentiation of stem-like cells toward a particular cell type (i.e. decrease the stem cell pool) but will do so specifically via change of cell fate from stem-like to mature by inhibiting fate-maintaining anti-cuRs.

This approach is fundamentally different from conventional regenerative medicine-related strategies that have largely concentrated on methods that manipulate the fate of stem-like cells at the level of ligands (e.g. cytokines), receptors, in vitro culture conditions, and signaling pathways. That is, the present application enables a method to manipulate the fate of stem-like cells and certain of their maturing non-stem cell progeny at key points downstream of ligand-receptor signaling, i.e. at points where fate decisions are ultimately made (i.e. at untranslated/noncoding RNA:RNA or RNA:DNA interactions), thereby short-circuiting/disengaging any would-be contributions by more upstream and potentially confounding ligand-receptor signaling pathways.

With regard to applications in the cancer space, the present invention teaches how the cuR targets can be readily procured and drugs/reagents readily developed therefrom for the purpose of manipulating the fate of stem or cancer stemline cells or possibly certain of their maturing non-stem cell progeny in vitro, ex vivo, or in vivo for experimental study or clinical use. Considering that current strategies in analysis and treatment of cancer fail to properly investigate or offer diagnostic and therapeutic targeting of the heretofore-elusive cancer stemline, the present invention will do both. More specifically, the present invention enables the development of novel reagents/drugs which will decrease the number of precursor cells (i.e. eradicate the stem cell pool: the cancer stemline) via induction of a permanent cell fate change from stem-like to mature/mortal by inhibiting fate-maintaining anti-cuRs thereby permanently eradicating a tumor's evasive stemline but sparing normal stem cells because stemline cuRs and normal stem cell cuRs are different. Normal stem cells and a cancer stemline harbor different cuRs in part because normal stem cells by virtue of dividing asymmetrically non-randomly maintain their leading DNA strands while regularly segregating out their lagging DNA strands to non-stem cell daughters, versus a cancer stemline which (by virtue of dividing symmetrically) randomly segregates its leading/lagging strands to its progeny.

In this way, the cuR expression pattern of normal stem cells (sustained by the reliable maintenance of strand segregation fidelity) differs from the cuR expression pattern of a cancer stemline (which lacks such strand segregation fidelity, and thus expresses cuRs different from those of normal stem cells). More specifically, and as an example, snoRNA-type fate-maintaining anti-cuRs will constitute targets against a cancer stemline (for the purpose of forcing a fate change to mortality) but will not be targetable against normal stem cells of that tissue type since normal stem cells, by virtue of ridding themselves of lagging strands (which will, however, be present in a cancer stemline) do not themselves (i.e. normal stem cells) permit tRNA-type sense-cuRs, which rely on lagging strand gaps for activity, to change their fate. Thus, therapies that block upstream snoRNA-type cuRs will be harmless to normal stem cells but toxic to a cancer stemline. There are other examples of this phenomenon arising from asymmetric versus symmetric segregation of factors leading to targetable differences between normal stem cells and a cancer stemline.

### Manipulation of cuR a Affect Not Only Stem-Like Cells but Also Maturing Non-Stem Cells

Since those progeny of stem-like cells which change their fate (i.e. maturing non-stem cell progeny) inherit, from their stem-like precursors, certain cuRs or mechanisms that resolve cuR:cuR (dsRNA) interactions (e.g. other stem-like features, such as antigens, are known to be inherited by maturing cells), attempts to manipulate stem-like cells by intervention with cuRs or mechanisms which act upon cuRs will also affect non-stem cells. Accordingly, manipulations of the types mentioned in relation to regenerative biology/medicine may affect not only stem cell pools but also pools of certain of their mortal maturing non-stem cell progeny. Similarly, manipulations of the type mentioned in relation to cancer may affect not only stem cell pools (i.e. stemlines) but also certain of their maturing non-stem cell progeny (i.e. faster-growing cancer cells).

While cuR-related actions occur not only at the level of the stem cell and its immediate daughters, but also at the level of maturing non-stem cells having inherited such a process from their stem like precursor cells, the actual sequences (i.e. identity) of particular cuR species are unique to both cell type (i.e. stem cell versus maturing non-stem cell) as well as tissue type. That is, the sequence or identity of cuRs acting in a stem cell in the liver is different from cuRs acting in a stem cell in the lung which, in turn, is different from ones acting in a non-stem cell in the lung. Moreover, the sequence (i.e. identity) of cuRs vary at different developmental transition points—e.g. Tsix (an anti-cuR) and Xist (a sense-cuR) act specifically at the embryonic stem cell stage whereas other cuRs of different sequence identity act in maturing non-stem cell daughters of embryonic stem cells as such cells undergo further fate transitions into more differentiated germ and somatic tissues. In this way, while the machinery which resolves cuR-related processes may be generally active in many cell types, the sequence of interacting cuRs will be quite specific to a particular cell and tissue type. It is also possible that, at times, the mechanism is active only in immature stem-like immortal-like cells and not in the more mature mortal-like cells.

Thus, an array of cuR-type sequences exist, the procurement of which provides specific targets for which to manipulate cell fate decisions at the level of stem cells, progenitor cells, and more mature cells in multiple tissue types for the purpose of studying or treating conditions relating to regenerative biology/medicine, cancer. Since cuRs will be specific to cell and tissue type, particular cells can be targeted while others left unaltered. Also, since cuR identities will be distinct in normal stem cells versus a cancer stemline derived from the same tissue, cancer stemlines can be targeted while normal stem cells left untargeted thereby limiting toxicity.

It should also be noted that, as previously mentioned, independent invention of tactics which affect the fate of mature non-stem cells (e.g. for the purpose of studying or treating conditions relating to regenerative biology/medicine or cancer) that do so because such tactics affect cuRs or the (nucleic acid, protein, riboprotein, or other molecule-based) mechanisms by which cuR:cuR interactions are resolved should be considered infringements upon the teachings of this application, even if such tactics do not directly affect stem-like cells themselves or their particular cuR sequences.

In other words, and with specific regard to novel anticancer measures, eradication of a cancer stemline via intervention with the cuR-related mechanism as taught herein (i.e. at the level of the nucleic acids which will or have paired, or at the level of nucleic acids, proteins, riboproteins, or other molecules acting upon said paired structure) may also significantly eradicate fast-growing cancer cells and thus lessen tumor bulk as an added benefit (e.g. as would be the case if the therapy non-specifically attacks cuR-related mechanisms or cuRs present in both the stemline and fast-growing cells—i.e. therapies designed to certain conserved regions of cuRs).

Thus one independently inventing measures that diminish tumor bulk by affecting cuRs or mechanisms which resolve sense-cuR:anti-cuR interactions should not a priori assume that one's intervention is not primarily stemline-directed. Such an intervention is indeed stemline-directed as taught herein simply with added benefits of also lessening tumor bulk (i.e. killing both fast-growing as well as slow-growing cancer cells since cuR-type mechanisms while at times present in fast-growing cancer cells are ultimately derived/inherited from the founder cancer stemline). Whether or not such tactics kill the stemline is not the issue as the identity/sequence of certain cuRs is different between the stemline and fast-growing cells but the mechanism which resolves such is inherited by the latter from the former as taught by the present invention.

Of note, differentiation-related therapeutic strategies (77A), while of benefit in certain rare cancers (i.e. acute promyelocyte leukemia), still have not proven clinically useful for the more common (solid) cancer types. One reason is that, as taught herein, differentiation therapies (e.g. retinoids) largely spare the cancer stemline of proper targeting because such treatments target upstream signaling pathways that are preferentially expressed by non-stem cells in tumors and thus spare the stemline—also such non-stem cells express such in a varied/heterogeneous fashion (because of genetic/epigenetic alterations) and thus are not all susceptible to such therapy, are preferentially expressed by non-stem cells in tumors and, even if non-stem cells express such in a homogeneous fashion, the stemline is still spared, or even if the cancer stemline is targeted in this way, the stemline is not eradicated because it simply is forced (by this therapy) to produce differentiated progeny, but can still simultaneously renew itself—thus is not fully eradicated. The teachings of the present application, unlike differentiation-related therapies, detail methods to truly eradicate the stemline.

### Designing or Screening for Drugs that Manipulate cuR Targets

Since the cuR targets are RNA-based (i.e. RNAs or riboproteins), reagents/drugs developed to interfere with/inhibit such will accordingly consist largely of classes of reagents/drugs which are RNA-affecting/binding. Such classes of reagents/drugs may include: ribozymes, antisense, dsRNA, antibodies to RNA or dsRNA, protein nucleic acids (PNA's), small molecules that affect/bind RNA, or other RNA-affecting/binding molecule. The development of such reagents/drugs is readily enabled by the present invention. Such development directly follows from procurement of cuR targets, which too is readily enabled by the present invention. As will be shown, development of the reagents/drugs can be readily achieved through knowledge of the sequences of procured cuRs by methods well-known to those skilled in the art by the following methods: intelligent custom-design of antisense or ribozyme reagents/drugs tailor-made to bind a specific cuR target RNA sequence (70), or high throughput screen of libraries of RNA-based or non-RNA-based small molecules that can affect/bind RNA targets (70, 73-76, 78). Also, reagents/drugs can be designed or screened for in similar manners to target those molecules in the fate-determining mechanism that are proteins, riboproteins, nucleic acids, or other molecular species.

That is, following procurement of cuR-type species and determination of their sequences, intelligent design of RNA-based therapeutics (e.g. antisense, ribozyme) can proceed in well-described manners (70). Also of note, in addition to RNA-based reagents/drugs, small molecule reagents/drags that specifically affect/bind RNA targets have also been described, and libraries thereof are commercially available or can be readily designed (73-76). Such libraries can then be screened for those small molecule species which 1) best affect/bind a given cuR target RNA sequence, e.g. by molecular assay, or 2) best produce the desired phenotypic result (i.e. maintenance or change of cell fate), e.g. by cellular assay, via affecting/binding i) a given cuR target sequence, or ii) an unknown cuR target sequence (e.g. from a collection of procured potential cuR sequences, as will be explained).

It should also be noted that development of the (RNA- or small molecule-based) reagents/drugs can also be performed without specific knowledge of cuR target sequences (e.g. via construction of a reagent/drug library to a collection of potential cuR sequences). That is, a collection of procured cuR species can be transformed into a library of species complementary (i.e. antisense) to the collection of cuRs (e.g. by single strand replication). Such a library (of cuR complements) can then serve as a reagent/drug library for which to screen, in a high throughput manner, for the purpose of finding which of such antisense species within the library has therapeutic effect (i.e. can affect cell fate) by blocking its corresponding complementary fate-determining cuR (from which it was constructed). Use of a molecular or cellular assay system as a basis for this reagent/drug screen will allow antisense reagents/drugs from such a library to be identified which either block anti-cuRs' (i.e. change fate) or block sense-cuRs (i.e. maintain fate). The corresponding cuR targets of those identified antisense reagents/drugs can then be determined by appreciating the reverse complementary sequence of the antisense reagent/drug identified. In this way, determination of an efficacious reagent/drug and its corresponding target sequence will enable further optimization of potential lead compounds by manners well known to those skilled in the art of intelligent design of RNA-based or small molecule pharmaceuticals (70, 73-76).

Reagent/drug libraries of an RNA-based or non-RNA-based, e.g. small molecule, nature can then be screened in a high throughput way, e.g. via cuR target binding competition assays, functional assays. With regard to functional assays, certain reliable systems can be readily employed to test the potential efficacy of a given RNA- or small molecule-based reagent/drug that is cuR affecting (i.e. fate-maintaining or a fate-altering). More specifically, such assay systems may include stem-like cell systems (normal or neoplastic) capable of differentiation-induction (i.e. fate change), as will be described. More specifically, constructs with detectable readouts (e.g. fluorescence) can be designed to report the reagent/drug-induced change in activity of a given cuR target. For example, reagent/drug interference with the anti-cuR Tsix species will effect a downstream reportable readout characterized by increased expression of Xist and decreased expression of certain X-linked genes.

An X-linked green fluorescent protein (GFP) construct will reflect such changes in a readily detectable and high throughput manner (79). Other constructs can be built to report expression changes arising at other allelic interaction sites (e.g. imprinted 11p or 15q regions, lymphoid recombination-related sites). For example, reporter artificial chromosomes to some of these regions have already been described (80), others can certainly be readily constructed by those skilled in the art. Additionally, an 11p-linked GFP construct will reflect reagent/drug-induced changes from biallelic 11p-linked expression (in a cancer stemline) to monoallelic 11p-linked expression—a change that would be desirable (and possible via targeting of an upstream anti-cuR expressed by the stemline) for the purpose of eradicating such a stemline. Such would appear grossly as a change from biallelic 11p-linked expression, i.e. loss-of-imprinting (LOI), to monoallelic 11p-linked expression, i.e. imprinting, in not only the stemline but also certain of the stemline's faster-growing more numerous and thus more readily detectable progeny thereby qualifying as a desirable assay system for high throughput screening. In these and other related ways, RNA-based and small or large molecule-based reagent/drug libraries can be screened in a high throughput manner for the purpose of identifying compounds within the libraries that can affect fate-determining cuR targets and their more downstream and potentially more readily detectable readouts for the purpose of studying or treating conditions relating to regenerative biology/medicine or cancer. Also, small or large molecule-based reagent/drug libraries can be screened in a high throughput manner for the purpose of identifying compounds within the libraries that can affect those molecules within the fate-determining mechanism which are proteins, riboproteins, nucleic acids, or other molecular species for the purpose of studying or treating conditions relating to regenerative biology/medicine or cancer, or other.

### Procurement of Anti-cuR and Sense-cuR Targets

As stated, it is proposed that cells maintain fate via either 1) preferential expression of an anti-cuR species which blocks a fate-altering sense-cuR species, or 2) preferential expression of a mechanism (which includes nucleic acids, proteins, riboproteins, or other molecules) which modifies/degrades an anti-cuR:sense-cuR complex which when not degraded, e.g. in a cell of changed fate wherein the mechanism is not expressed, leads to a fate change. In this way, by the former, it is proposed that amid a total cellular RNA population there exists 1) a subpopulation of RNAs (called anti-cuRs) which is differentially expressed and thus preferentially active in some daughter cells to maintain fate, as well as 2) a separate subpopulation of RNAs (called sense-cuRs) which is differentially active (e.g. because not postranscriptionally repressed by an anti-cuR) in other daughter cells to change fate. It should be aptly noted that such cuRs may act as stand alone single-stranded RNAs (ssRNAs) or in combination with other molecular species (e.g. proteins) as combined entities (e.g. riboproteins). Thus, some cuRs exist largely as 1) riboproteins, while others as 2) ssRNAs (short-lived in this state) that readily form 3) RNA:DNA species (e.g. fate-altering or -maintaining sense-cuR:DNA complexes), or 4) RNA:RNA (dsRNA) complexes (e.g. fate-maintaining or -altering sense-cuR:anti-cuR complexes) which in turn are readily 5) modified or degraded by dsRNA-mediated modification or degradation processes such as RNA-editing (RNA-E), RNA-interference (RNA-I), or PKR-related programs.

In this way, if the mechanism which modifies/degrades an anti-cuR:sense-cuR complex is differentially expressed in cells of maintained (or changed) fate, it is predicted that modified/degraded products of dsRNAs will be preferentially found in cells of maintained versus changed fate (or vice versa), and fate-altering RNA:DNA complexes will be preferentially found in cells of changed versus maintained fate (or vice versa).

Accordingly, and as will be detailed, strategies to procure cuRs derive from knowledge and exploitation thereof of the life cycle of such (i.e. whether existing as riboproteins, ssRNAs, RNA:DNA's, RNA:RNAs, or modified/degraded products of RNA:RNAs). As will also be detailed, RNAs of suspected cuR-type homology isolated in the manners can then be verified of such by 1) direct sequencing, or 2) prior to sequencing, first refinement/purification of procured clones to be sequenced via use of hybridization probes or polymerase chain reaction (PCR) primers with subjected cuR-type homology. Clones may also be refined via subtraction hybridization-type techniques (81), e.g. between cells of maintained versus changed fate, wherein anti-cuRs are preferentially active in cells of maintained fate whereas sense-cuRs are preferentially active in cells of changed fate.

With regard to cuR life cycles, untranslated/noncoding RNAs such as Tsix or Xist are expressed as ssRNAs, and the latter (Xist) is capable of forming RNA:DNA complexes with chromosomal DNA in cells of changed fate (38). In cells of maintained fate, however, Xist forms a dsRNA structure with its complement Tsix thereby temporarily preventing Xist from binding DNA and changing fate (or, possibly, Tsix blocks Xist action at the DNA-level, e.g. via enhancer/promoter inhibition) (16, 32)—such a dsRNA structure is short-lived (as is the case for other well-described dsRNAs) and then quickly modified or degraded thereby more permanently preventing Xist's fate-changing potential.

The dsRNA-related modification can be of an RNA-editing (RNA-E)-type wherein certain bases undergo transitions (e.g. adenosine-to-inosine, cytosine-to-uracil) (82-84). Alternatively, the dsRNA-related degradation may be of an RNA-interference (RNA-I)-type wherein such dsRNAs become truncated into 20-25 bp fragments (85-87).

As mentioned, Tsix would correspond to a particular anti-cuR which maintains the fate specifically of (embryonic) stem cell daughters of unchanged fate, whereas Xist would correspond to a particular sense-cuR which changes the fate specifically of (embryonic) stem cell daughters of changed fate (i.e. maturing non-stem cell daughters destined to differentiate further). As appears clear, Tsix is preferentially active in cells of maintained fate as a result of its differential expression or the differential expression other cuRs upstream of Tsix which maintain Tsix expression transcriptionally or post-transcriptionally (e.g. via blocking an inhibitor of Tsix, i.e. blocking an anti-Tsix with an anti-anti-Tsix). Also, as noted previously, a host of potential cuRs (e.g. in addition to Xist and Tsix) appear to reside within this region (39, 40), and thus may interact or modulate each other's action.

As another example previously noted, tRNA-type and snoRNA-type cuRs also function as key cell fate-determinants—the former (tRNA-types) as sense-cuRs, the latter (snoRNA-types) as anti-cuRs. In this way, both are initially expressed as ssRNA species (although intramolecular folding may produce localized dsRNA structures). tRNA-type sense-cuRs may then complex with DNA (as a primer for reverse transcription for some parasitic/retroelement or non-parasitic entity) (57, 65), thereby forming a fate-altering (or fate-maintaining) RNA:DNA complex in daughters that change fate.

In daughters that maintain (or change) fate, however, a snoRNA-type anti-cuR (or other cuR, antisense to the tRNA-type sense-cuR, e.g. not necessarily snoRNA-like) may form a dsRNA complex with the tRNA-type sense-cuR (i.e. snoRNA-type:tRNA-type complex) thereby preventing the tRNA-type sense-cuR from acting and thus in effect maintaining (or changing) cell fate. This dsRNA structure may then succumb to modifications such as RNA-editing (RNA-E) or RNA-interference (RNA-I), as will be described. Of note, certain snoRNAs are indeed known to bind RNAs (more specifically, rRNAs) and cause base modifications (66, 66A).

Thus, and as alluded to, procurement of cuRs derives from isolation of riboproteins, RNA:DNA, RNA (ssRNA and dsRNA), or modified/degraded dsRNA byproducts from the appropriate cell type under appropriate conditions with or without subtraction hybridization-type techniques. For example, stem-like cells grown under fate-maintaining conditions are expected to express anti-cuRs that are either riboproteins, ssRNAs, dsRNA complexes, modified/degraded dsRNA byproducts, or RNA:DNA complexes. On the other hand, stem-like cells grown under fate-altering (e.g. differentiation-induced) conditions are expected to express sense-cuRs that are either riboproteins, ssRNAs, within RNA:DNA complexes or dsRNA complexes, or modified/degraded dsRNA byproducts, in this way, 1) total cellular protein should include those anti-cuR and sense-cuR RNAs which exist as riboproteins, 2) total cellular DNA should include those RNAs complexed to DNA (i.e. RNA:DNA complexes) preferentially formed by sense-cuRs in cells of changed (or maintained) fate, and 3) total cellular RNA should include i) ssRNA (preferentially anti-cuRs in cells of maintained fate, and sense-cuRs in cells of changed fate), ii) dsRNA (anti-cuR sense-cuR complexes) preferentially in cells of maintained (or changed) fate, and iii) modified/degraded dsRNA byproducts also preferentially in cells of maintained (or changed) fate.

Accordingly, by techniques well-known to those skilled in the art, total cellular protein, DNA, and RNA can be isolated from a cell population (e.g. stem-like cell culture system) grown under non-differentiation-inducing (i.e. non-fate-altering) conditions wherein it is expected that an anti-cuR will be preferentially active, as well as from the same cells grown under differentiation-inducing (i.e. fate-altering) conditions wherein it is expected that sense-cuRs will be preferentially active. A number of differentiation-inducing agents/techniques are known—e.g. retinoids, sodium butyrate, histone deacetylators, cell starvation. The stem-like cell culture systems may include stem-like cells (e.g. embryonic stem cells, hematopoietic stem cells, neurologic stem cells), as well as tumor cell lines with stem-like properties or ones which can be induced to differentiate (e.g. teratocarcinomas, certain leukemic cell lines, solid tumor cell lines), as well as other cell line systems which can be induced to change fate/differentiate.

### Procurement of cuR-Type Riboproteins

With regard to procuring cuR species that are riboproteins, total cellular protein can be extracted via standard methods from those aforementioned cell systems under fate-maintaining versus fate-changing conditions. Subsequent treatment of such with proteinases will leave behind for further analysis only the RNA moieties that were previously protein-bound. Such RNAs could then be subjected to subtraction hybridization-type techniques (81), wherein those RNA moieties of an anti-cuR-type are preferentially derived from cells of maintained fate, whereas those RNAs of a sense-cuR-type are preferentially derived front cells of changed fate. Such RNAs could then be identified (before or after such subtraction) by 1) reverse transcription and mass sequencing of all clones, 2) reverse transcription and cDNA library construction using appropriate hybridization probes (of suspected cuR sequence homology) followed by sequencing of clones, or 3) reverse transcription polymerase chain reaction (RT-PCR) using appropriate primers (of suspected cuR sequence homology) followed by sequencing of clones. It should be noted that since such cuR-type RNAs are by definition untranslated/noncoding standard techniques of reverse transcription using oligo dT primers may have missed such RNAs (since, by virtue of being untranslated, may often not be polyadenylated and thus not well-represented in available microarrays, cDNA libraries).

Accordingly, reverse transcription with random priming may be the preferred technique for such efforts. Other techniques of riboprotein procurement and sequencing of RNA moieties are certainly also possible. Additionally, RNAs (or cDNA) procured in these manners can not only be probed/sequenced but also themselves used as probes (e.g. for microarrays, cDNA libraries, RNA/Northern analyses).

### Procurement of cuR-Type RNAs that are ssRNAs

With regard to procuring cuR species which are present as ssRNAs, total cellular RNA can be extracted via standard methods from those aforementioned cell systems under fate-maintaining versus fate-changing conditions. Subsequent treatment of such with RNases that destroy all RNAs except those that are ssRNAs may or may not be considered. Such ssRNAs could then be subjected to subtraction hybridization-type techniques (81), wherein those RNAs of an anti-cuR-type are preferentially derived from cells of maintained fate, whereas those RNAs of a sense-cuR-type are preferentially derived from cells of changed fate.

Such RNAs could then be identified (before or after such subtraction) by 1) reverse transcription and mass sequencing of all clones, 2) reverse transcription and cDNA library construction using appropriate hybridization probes (of suspected cuR sequence homology) followed by sequencing of clones, or 3) reverse transcription polymerase chain reaction (RT-PCR) using appropriate primers (of suspected cuR sequence homology) followed by sequencing of clones.

It should be noted that since such cuR-type RNAs are by definition untranslated/noncoding, standard techniques of reverse transcription using oligo dT primers may have missed such RNAs (since, by virtue of being untranslated, may often not be polyadenylated and thus not well-represented in available microarrays, cDNA libraries). Accordingly, reverse transcription with random priming may be the preferred technique for such efforts. Other techniques of ssRNA procurement and sequencing of RNAs are certainly also possible. Additionally, RNAs (or cDNA) procured in these manners can not only be probed/sequenced but also themselves used as probes.

### Procurement of cuR-Type RNAs Bound to DNA (in an RNA:DNA Complex)

With regard to procuring cuR species that are present in RNA:DNA complexes, total cellular DNA can be extracted via standard methods from those aforementioned cell systems under fate-maintaining versus fate-changing conditions (or vice versa). Subsequent treatment of such with RNase A (to destroy contaminating RNAs unbound to DNA), then DNase I (to destroy DNA) should leave intact only the RNA that was previously DNA-bound (such RNA is sensitive to RNase H, as a control) (89). Such RNAs could then be subjected to subtraction hybridization-type techniques (81), wherein those RNAs of a sense-cuR-type are preferentially derived from cells of changed (or maintained) fate.

Such RNAs could then be identified before or after such subtraction by 1) reverse transcription and mass sequencing of all clones, 2) reverse transcription and cDNA library construction using appropriate hybridization probes of suspected cuR sequence homology followed by sequencing of clones, or 3) reverse transcription polymerase chain reaction (RT-PCR) using appropriate primers of suspected cuR sequence homology followed by sequencing of clones. It should be noted that since such cuR-type RNAs are by definition untranslated/noncoding, standard techniques of reverse transcription using oligo dT primers may have missed such RNAs since, by virtue of being untranslated, may often not be polyadenylated and thus not well-represented in available microarrays, cDNA libraries. Accordingly, reverse transcription with random priming may be the preferred technique for such efforts. Other techniques of RNA:DNA procurement and sequencing of RNAs are certainly also possible. Additionally, RNAs or cDNA procured in these manners can not only be probed/sequenced but also themselves used as probes.

### Procurement of cuR-Type RNAs that are dsRNAs

With regard procuring cuR species which are present as dsRNAs, total cellular RNA can be extracted via standard methods from those aforementioned cell systems under fate-maintaining versus fate-changing conditions (or vice versa). Subsequent treatment of such with RNases that destroy all RNAs except those that are dsRNAs may or may not be considered. Such dsRNAs could then be subjected to subtraction hybridization-type techniques (81), wherein those RNAs of an anti-cuR-type are preferentially derived from cells of maintained (or changed) fate (wherein dsRNA structures form). Such RNAs could then be identified (before or after such subtraction) by 1) reverse transcription and mass sequencing of all clones, 2) reverse transcription and cDNA library construction using appropriate hybridization probes (of suspected cuR sequence homology) followed by sequencing of clones, or 3) reverse transcription polymerase chain reaction (RT-PCR) using appropriate primers of suspected cuR sequence homology followed by sequencing of clones. dsRNAs may also be retrieved via the use of dsRNA binding proteins (dsRNABP's).

It should be noted that since such cuR-type RNAs are by definition untranslated/noncoding standard techniques of reverse transcription using oligo dT primers may have missed such RNAs since, by virtue of being untranslated, may often not be polyadenylated and thus not well-represented in available microarrays, cDNA libraries. Accordingly, reverse transcription with random priming may be the preferred technique for such efforts. Other techniques of dsRNA procurement and sequencing of RNAs are certainly also possible. Additionally, RNAs or cDNA procured in these manners can not only be probed/sequenced but also themselves used as probes.

### Procurement of cuR-Type RNAs that have Undergone dsRNA-Mediated Modification/Degradation

With regard to strategies for procuring cuR species which are modified/degraded dsRNA byproducts, it has been shown that dsRNAs are special in that such are often rapidly modified or degraded in certain ways: e.g. via RNA-editing (RNA-E), RNA-interference (RNA-I), or PKR-related processes. Of particular note, the products of RNA-E and RNA-I are potentially retrievable. The PKR-related mechanism is more non-specific (i.e. dsRNA is degraded by PKR in a less regular and readily retrievable fashion) (90).

Accordingly, in order to remove the potentially confounding contributions by such PKR-related programs to dsRNA processing thereby allowing efficient procurement of cuR species that have undergone RNA-E-type or RNA-I-type processing, the PKR-related mechanisms could be intentionally inhibited when assaying for dsRNAs or their modification/degradation products, as previously advised (17). Such may not even be necessary especially if PKR-related processes are preferentially active in mature but not immature stem-like cells that are maintaining fate. Alternatively, PKR-related processes may be active in immature stem-like cells but only with respect to certain RNA species (e.g. longer ones) while leaving others (e.g. short RNAs) alone (12).

RNA-editing (RNA-E) is a process wherein dsRNA structures are modified such that certain bases are changed from usual to unusual (e.g. adenosine-to-inosine, cytosine-to-uracil) (82-84). This process may occur in mRNA thereby leading to altered messages and protein structures—and such, at times, may for as yet unknown reasons constitute a necessary part of the life cycle of certain proteins (82). RNA-E also acts as a defense system against certain viruses (83, 84). In these cases, it appears that the editing of bases takes place preferentially in the nucleus thereby leading to nuclear retention and thus translation inhibition of certain virally-derived RNAs. Accordingly, certain cuR-type sequences that form dsRNAs (anti-cuR:sense-cuR complexes), e.g. Tsix:Xist or tRNA-type:snoRNA-type, may similarly undergo base modifications that prevent the action of the sense species (e.g. Xist, or tRNA-type, and others) to change (or maintain) fate. In other words, in this case the anti-cuR (e.g. Tsix, snoRNA-type, and others) by binding the sense-cuR and causing base modification (via RNA-E) prevents the sense-cuR from effectively binding DNA to cause a fate change (or maintenance). If such cuRs rapidly form dsRNAs and rapidly undergo RNA-E, then the most effective method of procuring such for the purpose of identifying them by sequencing may be to concentrate on procuring the edited species rather than on the short-lived ssRNA or dsRNA species.

Procurement of edited cuRs may be optimized via RNA purification techniques that preferentially isolate RNAs that are nuclear-located—e.g. via purification of cellular nuclei (i.e. separation from cytoplasm and cytoplasmic RNA) and isolation of nuclear RNA therefrom. The use of dsRNA binding proteins (dsRNABP's) to fish out nuclear RNAs that are double-stranded may also be helpful. Also, the use of novel probes/primers specifically designed to hybridize to edited bases (e.g. inosines) are expected to secure edited RNAs not yet well-detected by conventional techniques which use probes/primers that do not hybridize well with such unusual bases (83, 84).

RNA-interference (RNA-I) is another process by which dsRNAs are resolved. More specifically, as has been recently well-documented, RNA-I is a defense system against dsRNA sequences deemed to be parasitic or potentially fate-threatening and involves the degradation of such dsRNAs into small 20-25 bp fragments, mostly localized to the cytoplasm (85-87). Of note, small RNA fragments which appear to have arisen from RNA-I-type activities have been found not only in association with degradation of parasite-derived sequences but also endogenous developmentally-related sequences (11, 12, 47, 91). Accordingly, certain cuR-type sequences that form dsRNAs (anti-cuR:sense-cuR complexes), e.g. Tsix:Xist or tRNA-type:snoRNA-type, may similarly undergo RNA-I-type processing that prevents the action of the sense species (e.g. Xist, or tRNA-type, and others) to change (or maintain) fate. In other words, in this case the anti-cuR (e.g. Tsix, snoRNA-type, and others) by binding the sense-cuR and causing its degradation (via RNA-I) prevents the sense-cuR from effectively binding DNA to cause a fate change (or maintenance).

If such cuRs rapidly form dsRNAs and rapidly undergo RNA-I, then the most effective method of procuring such (for the purpose of identifying them by sequencing) may be to concentrate on procuring the degraded (small 20-25 bp) cytoplasmically-located species (rather than on the short-lived ssRNA or dsRNA species). Procurement of small degraded cuRs may be optimized via RNA purification techniques that preferentially isolate RNAs that are small—e.g. via techniques previously described (85-87). That is, it has been argued that such small RNAs are not readily procured by standard RNA isolation techniques so may require special efforts to find, as noted (85-87). The use of dsRNA binding proteins (dsRNABP's) to fish out RNAs that are double-stranded may also be helpful. Moreover, recent demonstration of the existence of mammalian homologs of Dicer (a dsRNase that yields small RNA byproducts) may allow the use of Dicer, Dicer-related, or other nuclease complexes to similarly fish out dsRNAs prior to their degradation via RNA-I-type processes (11, 91-93).

As alluded to, RNAs having undergone modifications/degradations involving RNA-E-type and RNA-I-type processes have escaped standard detection techniques due to 1) the difficulty of hybridization techniques to detect edited bases, 2) the inefficient isolation of very small RNAs by standard RNA procurement techniques. Accordingly, special attention to such, as detailed in the present application, will allow one to more efficiently procure such. Specifically, following procurement of RNA fractions having undergone RNA-E-type or RNA-I-type processing, as previously described, such fractions can then be subjected to 1) reverse transcription (using random priming) and mass sequencing of all clones, 2) reverse transcription and cDNA library construction using appropriate hybridization probes of suspected cuR sequence homology followed by sequencing of clones, or 3) reverse transcription polymerase chain reaction (RT-PCR) using appropriate primers of suspected cuR sequence homology followed by sequencing of clones. Alternatively, RNAs (or cDNA) procured in these manners can not only be probed/sequenced but also themselves used as probes, as previously described, e.g. for that fraction undergoing RNA-I (11).

### Differential Expression Not of cuRs but of the Mechanism that Resolves cuR-Related Action

As mentioned above, an alternative scenario may also be possible wherein certain anti-cuRs and sense-cuRs are not differentially expressed but rather are equally expressed in those daughters that maintain fate and those which change fate, but that the mechanism (which includes nucleic acids, proteins, riboproteins, or other molecular species) which resolves such dsRNAs is itself differentially expressed. In this way, daughter cells could maintain fate either by 1) preferentially expressing anti-cuRs thereby forming anti-cuR:sense-cuR complexes the modification or degradation of which prevents the sense-cuR from changing cell fate, or 2) preferentially expressing the mechanism that resolves dsRNAs that acts to modify or degrade such anti-cuR:sense-cuR complexes (i.e. such complexes could form also in daughters of changed fate but not be adequately modified or degraded thereby leaving sense-cuRs available to change fate).

On the other hand, daughters cells could change fate either by 1) preferentially not expressing anti-cuRs while expressing fate-altering sense-cuRs, or 2) expressing both anti-cuRs and sense-cuRs but preferentially not expressing the mechanism that resolves dsRNAs that acts to modify or degrade such anti-cuR:sense-cuR complexes thereby allowing sense-cuRs to, in an unimpeded way, change cell fate. In this way, one can undertake a strategy of differentially procuring the modified/degraded byproducts of anti-cuR:sense-cuR dsRNA interactions (e.g. following RNA-E-type or RNA-I processing) preferentially from cells that maintain (or change) fate. That is, the prediction being that while anti-cuRs are expressed in both cells of maintained as well as changed fate, the modified/degraded products of dsRNA formation will be preferentially found in cells of maintained (or changed) fate (where the dsRNA modification/degradation mechanism is most active).

### Analysis of Procured/Cloned Potential cuR-Type Species

As stated, those RNAs of suspected cuR-type homologies can be procured first by isolating the following cellular fractions, riboproteins, ssRNAs, RNA:DNA's, dsRNAs, and dsRNA byproducts. Such fractions can then be individually reverse transcribed (e.g. using random priming), cloned, and then:

1) used as probes themselves (11), e.g. against microarrays, cDNA libraries, RNA/Northern analyses, for the purpose of cloning related or full-length cuR-type species which can then themselves be sequenced, or:

2) directly mass sequenced and then compared with known or suspected cuR-type characteristics/motifs, e.g. such characteristics/motifs may include those of known noncoding RNAs or ones contained in databases such as: lack of a proper open reading frame (ORF) (e.g. multiple small ORF's) (94, 95), lack of a proper a polyadenylation (pA) signal or not well-compartmentalized despite possessing a pA signal (96), lack of many (if any) introns (97), transcribed from within an intron or from with some other noncoding region (e.g. enhancer, promoter) (98, 98A), harboring repetitive-type or parasitic-appearing sequences (97, 99, 100), small RNA size (101), sharing characteristics-motifs with other known untranslated RNA species as is found in certain databanks including noncoding RNAs, tRNAs, snoRNAs, (101-108), or sharing characteristics/motifs with other potential cuR-type sequences (some of which are listed below), or:

3) such fractions can be purified further via cDNA library screening or RT-PCR using probes and primers, respectively, of suspected cuR-type sequence homologies—the resultant clones of which can then be sequenced.

The following list, not meant to be exhaustive, includes some candidate sequences of suspected cuR-type homologies or may indeed constitute actual cuR sequences themselves—the sequence information from which may used to design probes/primer to procure cuR sequences by cDNA library screening, RT-PCR, cloning, and sequencing.

### EXAMPLES

The following are examples of fate-determining molecules. The fate-determining molecules include nucleic acids, proteins, riboproteins, other fate-determining molecules or combinations of molecules.

The examples in no way are intended to limit the scope of the invention, but are simply include as specific embodiments of the present invention.

The mechanism is made up of interacting nucleic acids, which are acted upon by a host of proteins and riboproteins. Examples of target fate-determining proteins be acted upon or used to change the fate of cells include: RdRp-like (e.g. qde1, sgs2, ego1), eIF2C-like (e.g. qde2, rde1, RecQ-like (e.g. qde3), RNase D-like (e.g. mut-7), piwi, hiwi (135), ADAR.

Also an example of a riboprotein that could be targeted in the fate-determining mechanism is the RNA component of the riboprotein RNase MRP (RMRP).

In addition, the following examples are based on RNA fate-determining molecules. The invention includes all RNA molecules acting as part of the fate-determining mechanism which may or may not be untranslated/noncoding and could derive from the cell genome, from cellular organelles such as mitochondria and more specifically tRNA and rRNA, or foreign RNAs, as well as other independently replicating RNA's.

### 1. Xist, Tsix

Xist is a sense-cuR that changes the fate of certain embryonic stem cell daughters (38). Tsix is an anti-cuR that maintains the rate of certain embryonic stem cell daughters (32). The novelty here is that the present invention teaches that such are targets for fate manipulations in the regenerative medicine and cancer spaces, for example, therapeutic inhibition of Tsix via use of an antisense, ribozyme, or small molecule drug would be predicted to eradicate a stemline within certain germ cell tumors. On the other hand, inhibition of Xist expands stem cell pools, e.g. for the purpose of regenerating germ cells. There are additional untranslated/noncoding RNAs in this region of the X-chromosome (39, 40), which also act as key cuR-type species in need of procurement and targeting for the purposes described.

### 2. S (Switch) Sequence

S sequence is a sterile transcript which forms an RNA:DNA complex and effects class switch recombination (34-37). It also has a complementary antisense species (anti-S) that regulates its action (i.e. prevents switching in more immature cells maintaining their rate). The S has parasitic-like features in that it contains repeat sequences 20-100 bp long and is derived from intronic (promoter) regions (34-37). For example, therapeutic inhibition of anti-S in certain lymphoid-derived cancers would be predicted to cause a stemline to assume a mortal phenotype. On the other hand, inhibition of the S sequence might be used to expand immature lymphoid cells, e.g. to boost immunity, for regeneration purposes.

### 3. Let-7 (and Other Related Family Members)

Let-7 is a small (21 bp) untranslated RNA species that possesses developmental function in multiple tissue types (109, 110). It may bind DNA (i.e form an RNA:DNA complex) thereby acting as a fate-changing sense-cuR. In this way, let-7 also possesses a complementary antisense species (anti-let-7) that regulates its action (i.e. prevents changes in cells maintaining their fate). Alternatively, let-7 may act as an anti-cuR—i.e. as an antisense to its proposed complement (sense-let-7) thereby maintaining fate by inhibiting the fate-altering action of sense-let-7. let-7 shares homology with certain other species such as the lin-4 transcript (22 bp), and thus may constitute one member of a much larger family of cuR-type species worthy of procurement and targeting not heretofore appreciated by those in the regeneration biology/medicine or cancer spaces. Of note, there is evidence that let-7 functions in the developing bone marrow, lung, and other tissues (109, 110), and thus may itself or its complementary antisense be a key cuR-type target in regeneration-related and cancer-related problems derived from these tissues. For example, if anti-let maintains the immortal fate of a stemline within a hematologic or lung cancer, then therapeutic inhibition thereof via antisense, ribozyme, small molecule, should eradicate the stemline.

### 4. kappaNE, kappaBS (and Other Enhancer-Related RNAs)

Kappa NE is a short RNA (27 bp) with homology to a parasitic-like repetitive element and lies within an intronic enhancer (50). That this RNA functions in RNA:RNA or RNA:DNA activities is as yet unclear, but it is interesting to note that it does negatively regulate certain fate changes (raising the possibility that it acts as a cuR). Also, another short RNA (i.e. kappaBS), with a short region of complementarity to kappaNE, abolishes the effect of kappaNE suggesting a possible but as yet to be shown antisense:sense (or possibly an RNA:DNA) interaction (50). In this way, interruption of such will have regeneration- and cancer-related ramifications in lymphoid tissues. Other enhancer region sequences with potential cuR-type activities include the pTa region with homology to Xist (49).

### 5. Neurologic-Associated cuR-Type Species (Disc1, Disc2, and C6orf4-6, C6UAS)

Disc1 and Disc2 are schizophrenia-associated loci that may undergo allelic communications as suggested by evidence that their translocation disrupts activity (111). C6orf4-6 and C6UAS are also schizophrenia-associated and share overlapping complementarity suggesting a possible fate-determining RNA:RNA interaction (e.g. of an anti-cuR:sense-cuR type) (112), and a potential target for regenerative- and cancer-related purposes for neurologic tissues.

### 6. Okazaki Fragment-Related cuR-Type Sequences

Of note, certain antisense sequences have been detected at Okazaki sites (68), suggesting a possible role for antisense regulation in lagging strand biology, as proposed in the present application. Moreover, that short regulatory RNAs do indeed act in lagging strand biology has been shown via demonstration that RNAs 10-15 bp in length can prime synthesis of Okazaki intermediates (67, 67A). Also, Okazaki fragments have been shown to be filled in by 21 bp fragments prior to their ligation (67A). Accordingly, the sequences in these aforementioned publications (67, 67A, 68) provide important and novel information for the construction of probes/primers used to procure cuR-type species. Such targets can then form the basis of a design or screen for regeneration- and cancer-related fate-manipulating reagents/drugs.

### 7. Fate-Determining tRNA-Type and snoRNA-Type cuR-Like Species

As mentioned previously, certain tRNA-type sequences may function as sense-cuRs which change fate (e.g. via priming reverse transcription of certain fate-altering elements, e.g. retroelements such as LINE-1). Such tRNA-type sequences may initiate such reverse transcription activities at lagging strand gaps, e.g. as a recombination/repair-type process (52, 53). Moreover, such tRNA-type sequences may closely resemble mito met tRNA—as suggested by the strong homology between human mito met tRNA and certain fate-altering tRNA-type sequences (59, 60). Other cuR-type tRNAs may include other mitochondrial tRNAs as well as certain nuclear tRNAs (102, 103). Clues as to which tRNA sequences may serve as key primers in this regard may be elucidated via appreciation of 1) those 18 bp recognition sites used in reverse transcription priming (57, 65), as well as 2) those tRNA-related sequences left as remnants following allele-exchange-type events (55), or to be left (e.g. in humans) as such if properly investigated (58). Of note, certain antisense sequences have been shown to interfere with tRNA function thus such may serve as (or assist in the construction of) reagent/drug equivalents of anti-cuRs (116, 117).

Also as mentioned previously, snoRNA-type sequences may function as anti-cuRs that block the fate-altering function of certain tRNA-type sequences (that prime fate-altering reversely transcribed elements, e.g. LINE-1). Such snoRNA-type sequences may closely resemble or be comprised of those that are allelically excluded (e.g. imprinted), some of which have been recently reported (63, 64). Other snoRNAs interact with rRNAs act a 10-21 bp recognition site (118, 119), which may provide important sequence information with which to procure snoRNA-type cuRs. Of note, there are a number of snoRNA species with no clear substrates (61-63), and it has been recently suggested that one such substrate might be tRNA-like (62) thereby indicating that certain snoRNA-type species may act as (and thus provide sequence information for the procurement of) anti-cuRs.

Interestingly, a key cancer-related connection between tRNA (mitochondrial) and snoRNA biology has recently been shown: the MRP riboprotein has an RNA-based moiety and acts in both mitochondrial and nucleolar strand synthesis, and alterations in this process are associated with some cancers (69, 70).

### 8. Miscellaneous

### Germ Cell-Related cuRs

A region of human chromosome 12, i(12p), as well as human chromosome 11q13 have been regularly reported in germ cell cancers (120, 121). There is a strong possibility that such harbor certain untranslated/noncoding RNAs that act as key cuRs which maintain the fate of germ cell cancer stemlines (i.e. anti-cuRs) and change fate of certain germ cell cancer cells as they differentiate (i.e. sense-cuRs). The anti-cuRs would constitute key targets to develop or screen for drugs to eradicate the stemline.

### Autosomally Imprint-Related cuRs

As proposed by the present application, given the preponderance of untranslated/noncoding antisense sequences at certain imprinted loci (16, 33), there are key fate-altering RNA:DNA (i.e. sense-cuR:DNA) events at autosomally imprinted regions in cells that change fate, as well as key fate-maintaining RNA:RNA (i.e. anti-cuR:sense-cuR) events at autosomally imprinted regions in cells that maintain fate. The identity of the sense-cuRs may be learned via methods described wherein RNA:DNA complexes are purified from cells of changed fate and then RNA alone procured. Such cuRs can be used to probe or used as probes themselves in conjunction with cloned imprinted regions (e.g. 11p, 15q, and other allelically excluded loci) in order to identify sense-cuRs in this region. Also, procurement of anti-cuRs in these regions may follow isolation of dsRNAs or dsRNA byproducts from cells of maintained fate to probe or use as probes in conjunction with cloned imprinted regions (e.g. 11p, 15q, and other allelically excluded loci).

### Lymphoid-Related cuRs

As previously stated, allelic exclusion preceding VDJ recombination involves a demethylation event (46). Considering the evidence front an array of systems that epigenetic activities that are of a methylation-type can be preceded by ones which are RNA-related (16, 122), it is evident that an allelic exclusion event is ultimately initiated by a sense-cuR:DNA event that is tonically inhibited in cells maintaining fate by an anti-cuR:sense-cuR interaction. Sequence information regarding such sense-cuRs and anti-cuRs can then be used to design primers/probes to procure such. Continuing on the lymphoid-related theme, the recent demonstration of a key role by RNA-editing in lymphoid development indicates that in addition to the editing of mRNA (123, 124), the editing of untranslated/noncoding dsRNAs (of an anti-cuR:sense-cuR type), as proposed in the present invention, may also be key.

### cuR Species when in the Form of ssRNA, RNA:DNA, or dsRNA

As stated, ssRNA, RNA:DNA, and dsRNA fractions isolated from cells of maintained versus changed fate can be not only (with or without subtractive hybridization-type techniques) 1) directly sequenced, 2) probed and primed via cDNA library screening and RT-PCR, respectively, with resulting clones sequenced, but also 3) used themselves as probes (11).

### cuR Type dsRNA and dsRNA Modified/Degraded Byproducts

As stated, use of dsRNABP's to fish out dsRNAs from cells of maintained fate will be helpful. Also, use of Dicer-related complexes to find dsRNAs prior to undergoing RNA-I-type processing will also be key (11, 91-93). Also of note, while RNA-I has been shown to act on parasitic elements, there is recent evidence that such may also act on endogenous (possibly developmentally-related) sequences as proposed by the present application (11, 12, 47, 91). Such sequences should provide important information for the design of probes/primers to procure human cuR-type species.

### Endogenous Small RNA Byproducts

It has been shown that certain endogenous small RNA byproducts may derive from retransposon sequences (12, 91). Other endogenous small RNA byproducts may be either retrotransposon or non-retransposon-derived (11, 47, 58).

The interruption of the fate-maintaining mechanism of the cancer stemline results in a change from stemline immortality to mortality and thus its eradication.

### A List of Specific cuRs

The following is list of cuRs is not intended to be comprehensive, but simply illustrative of cuRs that can be affected or used to change or maintain cell fate in the cell be treated: Tsix, Xist, or other X-linked untranslated/noncoding RNAs; Switch (S) sequences (in lymphoid regions) and their proposed antisenses; Let-7, Lin-4, (small RNAs) and their proposed antisenses; kappaNE, kappaBS (enhancer RNAs); untranslated RNAs involved in methylation or demethylation (e.g. involved in allelic exclusion prior to VDJ recombination); Disc1, Disc2, C6orf4-6, C6UAS (neurologic sense and antisense RNAs); Okazaki RNA primers, RNA intermediates of Okazakis, antisense sequences within or associated with Okazakis; certain fate-determining tRNA-type sequences (e.g. mito met tRNA-like, or primers of reverse transcription of either non-parasitic or parasitic, certain retroelements such as L1, origin); certain fate-determining snoRNA-type sequences (e.g. imprinted ones); untranslated/noncoding RNAs within germ cell cancer regions i(12p) and 11q13 untranslated/noncoding RNAs within certain imprinted regions (e.g. 11p, 15q) untranslated/noncoding RNAs within certain lymphoid-related allelic exclusions; untranslated/noncoding RNAs when riboprotein, ssRNA, RNA:DNA, or dsRNA; untranslated/noncoding RNAs when in the form of dsRNA modified/degraded products such as following RNA-interference or RNA-editing (e.g. small 21-25 bp RNAs, or RNAs with edited bp's); RNAs may be non-polyadenylated, lack open reading frames, lack introns, transcribed from within a noncoding region intron, enhancer, promoter, RNA:DNA and RNA:RNA, and DNA:DNA and other paired nucleic acid structure; all untranslated/noncoding RNA's from the host genome; all untranslated/noncoding RNA's from outside the host genome including organelles or independently replicating entities; proteins and riboproteins acting in the mechanism including but not limited to RdRp-like (e.g. qde1, sgs2, ego1), eIF2C-like (e.g. qde2, rde1, RecQ-like (e.g. qde3), RNase D-like (e.g. mut-7). (135), piwi, hiwi, RMRP (the RNA component of the riboprotein RNase MRP), ADAR; and satellite DNA, RNA; mitochondrial tRNA, mitochondrial tRNA-met; Y RNA, Air RNA, BC1 RNA; chromodomain proteins and riboproteins, chromomethylases with or without guide RNA's; mir142, and other human stRNA's; and snoRNA's, imprinted snoRNA's

### Examples of Treatments

One appropriate model system to use as an example is the germ cell cancer system. Germ cell cancers (testicular cancers, teratocarcinomas) are known to harbor stem-like subpopulations (125).

Moreover, it has been argued that certain embryonic-like features seen in these tumor types (e.g. expression of fetal antigens, or loss of imprinting/LOI) is a result not of dedifferentiate (as conventionally thought) hut rather of persistence of an embryonic-like phenotype in such tumors from the immature (e.g. stem-like) cells from which such tumors derive (126). The present application expands and improves on this point and teaches that those stem cells that give rise to tumors, in this case the germline stem cells, or primordial germ cells which give rise to germ cell cancers, actually persist in the tumors as its cancer stemline. In this way, the presence of embryonic-like features in these tumors is indeed not only a result of the persistence of such a phenotype from stem-like cells, but more specifically a result of the persistence of stem-like cells themselves as the tumor stemline in the tumor. Accordingly, as the stemline rears progeny which proliferative and invade, some of such progeny may undergo varying degrees of differentiation due to local differentiation-inducing signals—the somewhat dysregulated manner of which will lead to partially differentiated progeny wherein certain stem-like features (e.g. fetal antigens, loss of imprinting/LOI) are maintained in faster-growing cancer cells undergoing differentiation. Thus, the presence of embryonic-like feature in tumors (e.g. germ cell cancers) can be explained by the presence in tumors of a subpopulation of stem-like cells (i.e. a cancer stemline), and a larger population of immediate stemline progeny (faster-growing) cells undergoing partial differentiation that have inherited and thereby maintained and thus made more grossly detectable certain features of their founder precursor stemline (i.e. features which may not have been so readily detectable if present solely in the small stemline subpopulation and not passed on to more cells/bulk).

Other cancer types display the same phenomenon. That is, for example, certain tumors of the lung, breast, colon, and prostate show loss of imprinting/LOI at various loci (e.g. chromosome 11p, 15q, 1p, et al) (127-129). Such is expected to signify the presence of a cancer stemline and its immediate (and more numerous) partially differentiating cancerous progeny that have inherited certain stem-like features (e.g. loss of imprinting/LOI) thereby making such features more readily detectable.

Accordingly, the present application teaches that manipulations designed to eradicate a cancer stemline will lead, prior to extermination of the tumor, to a gradual loss (in the tumor) of embryonic features due to loss of the stemline and thus also its embryonic features, and loss of the stemline's immediate progeny and thus their inherited embryonic features. Such a readout will appear as a termination to the loss of imprinting/LOI—a.k.a. appearance of imprinting. That is, in a tumor those loci demonstrating LOI (i.e. biallelic expression, e.g. of Igf2, or biallelic silencing, e.g. of H19) will, following stemline eradication (or near eradication), switch to an imprinted expression pattern (i.e. monoallelic expression, e.g. of Igf2 or H19). This switch from LOI to imprinted expression will herald that the stemline is becoming increasingly mortal due to the therapeutic intervention and thus will provide an early readout for successful intervention prior to final eradication of the stemline.

Germ cell cancer stemlines also display LOI of certain X chromosome loci, i.e. the X chromosome is largely active (in an XY stemline of a male patient), in a female patient both XX's are active in a cancer stemline). Because of such, a readout is available wherein therapeutic manipulation of the stemline will initially cause one active X to become inactive prior to eradication of the stemline. Since certain faster-growing stemline progeny inherit this feature (i.e. an active X) from the stemline, the switch to an inactive X (thereby heralding successful manipulation of the stemline and near death of the stemline) will be readily detectable in the tumor.

It should be noted that the manipulation of a fate-determining mechanism in one cancer type may be specific to that particular cancer or may have broader relevance to other cancers also. For example the Tsix manipulations for testicular/germ cell cancers will also have benefit in other cancer types such as breast, prostate, colon which demonstrate alterations in X-chromosome status, namely biallelic expression or overexpression.

Targeting a fate-determining molecule, whether it is a nucleic acid, protein, riboprotein, other molecule or combination of molecules involved in the fate-determining mechanism. This mechanism may include specific examples X-linked Tsix-Xist, and RNA imprinted loci chromosome 11p and 15q.

### Experiments

Germ cell cancer cell lines are readily available commercially and through academic collaborations. One method of blocking the mechanism is to block the antisense RNA species that maintains stemline fate/immortality. Tsix is one such candidate antisense RNA species. Thus the plan is to design an artificial antisense RNA complementary/specific to Tsix (or other compound specific to Tsix, e.g. a ribozyme, double-stranded RNA) for the purpose of blocking Tsix function. Delivery of an artificial antisense to a germ cell cancer cell line results in inhibition of Tsix in the stemline, a readout switch from an active to an inactive X chromosome, cell death of the stemline and its progeny, loss of immortality of the cell line as determined by serial culturing.

In an in vivo mouse model, implantation of the cell line and treatment with chemotherapy will be improved upon by treatment not only with chemotherapy but also with the artificial antisense therapeutic (complementary to Tsix) that will have the added benefit of eradicating the stemline.

Also, in addition to blocking Tsix function, it is possible to overexpress Xist, by a transgene, in a cancer cell line in this way forcing the stemline to inactivate its X chromosome and thus assume mortality.

A related experiment has already been performed but in normal rather than neoplastic cells. More specifically, Lee has demonstrated that blocking Tsix function in embryonic precursor cells does indeed lead to their premature death (130). Such precursor cells give rise to and form the stemline of certain germ cell cancers. Accordingly, the blocking Tsix in such germ cell cancers (130), leads to stemline and cancer eradication. That such anti-cancer measures will spare normal stem cells of unwanted toxicity is indicated by other experiments by these investigators. That is, Tsix inhibition does not appear to be particularly problematic when directed to normal (embryonic) stem cells (131).

The mechanism is made up of interacting nucleic acids (RNA:DNA; RNA:RNA; DNA:DNA). These interacting nucleic acids are acted upon by a host of proteins and riboproteins, such as: RdRp-like (e.g. qde1, sgs2, ego1), eIF2C-like (e.g. qde2, rde1, RecQ-like (e.g. qde3). RNase D-like (e.g. mut-7). (135), piwi, hiwi, and RMRP (the RNA component of the riboprotein RNase MRP).

In this way, one desiring to interrupt the mechanism could act at the level of some of these or other proteins or riboproteins or at the level of nucleic acids for example, RNA. The invention includes not only protein-level interventions, but also protein-level interventions that directly or indirectly affect downstream fate-determining nucleic acid actions. For example, one can interfere with an interacting RNA species such as Tsix, or with a protein that functions in resolving dsRNAs such as qde1, hiwi, ADAR, or other.

Cell type specificity is afforded by the RNA sequences involved, as the proteins that act in this mechanism are the same from one cell or tissue type to another. It is possible that the entire mechanism (i.e. nucleic acids and proteins involved) are expressed solely in certain cell types (e.g. stem cells and not their more mature progeny), however it is also quite that the mechanism is inherited (in some part) by the latter from the former. While the same (or some) proteins may be inherited, there may be different RNAs involved depending on the maturity of the cell.

Also, while the application has stressed that this mechanism is stem cell and stemline-specific, it also mentions that certain non-stem cells (i.e. certain progeny of stem cells) may inherit the mechanism and thus use it too (but with slight variation, e.g. in sequence of interacting RNAs, or not). The point being that anti-cancer strategies targeting this mechanism may also kill fast-growing cells but this should not be interpreted as non-stemline-directed but rather stemline-directed with the added benefit of also killing fast-growing cancer cells.

All noncoding/untranslated RNAs are actually candidate targets (i.e. those RNAs which bind DNA or RNA to change or maintain cell fate, respectively). The application has suggested that these may be preferentially found at imprinted or allelically excluded regions (e.g. X-chromosome, imprinted 11p and 15q, lymphoid-related loci), but there are additional regions that harbor such (137), e.g. 1p36 wherein p73 lies. Also those RNAs that act at enhancer regions or are involved in RNA:DNA-mediated methylation changes as suggested prior to VDJ recombination (138) are also candidates.

Of note, a recent publication has found small 21-25 bp RNAs in mammalian cells (139, 140)—as predicted by the present application. The present application further predicts that such RNAs will include certain ones that have been degraded from dsRNAs the formation of which maintained (or changed) cell fate. Thus the sequence of such a small RNA will enable isolation of the interacting dsRNA and thus the sequence of a fate-maintaining (or changing) RNA that will serve as a novel target.

Also dsRNAs undergoing RNA editing are target candidates. Such may include certain tRNA-like and snoRNA-like species. Such RNAs may be genomically-derived or derived from other non-genomic entities (e.g. organelles such as mitochondria).

It is important to note the fate-determining mechanism includes sense and antisense RNA, other nucleic acids, proteins, riboproteins, and other molecules. Thus manipulation of these will assist in regenerative medicine-related measures seeking to expand and then contract stem-like cells to renew tissues.

Anti-cancer stemline measures may be accelerated somewhat by prior or concomitant induction of stemline differentiation using certain differentiation-inducing agents such as retinoids, histone deacetylase inhibitors. Indeed induction of differentiation of cells with altered mitotic mechanics have been shown to be forced to die (131).

It should be noted that the manipulation of a fate-determining mechanism in one cancer type may be specific to that particular cancer or may have broader relevance to other cancers also. For example the Tsix manipulations for testicular/germ cell cancers will have benefit in other cancer types such as breast, prostate, colon which demonstrate alterations in X-chromosome status, namely biallelic or overexpression. On the other hand some RNAs might affect just certain cancers.

Protein, riboprotein, and other molecular elements of the mechanism including but not limited to qde1, sgs2, ego1, eIF2C, qde2, rde1, RecQ, qde3, RNase D, mut-7 (135), piwi, hiwi, RMRP, and ADAR are targets for cell fate manipulation.

Since cancer stem cells and normal stem cells are different, differential targeting can be accomplished between the two. For example, cancer stem cells divide symmetrically thus equally segregate certain factors that include lagging strands to all daughters whereas normal stem cells do not (because such divide asymmetrically). Also, cancer stem cells are in larger abundance than normal stem cells which are rare thus dosing can be set in such a manner so as to limit effects on normal stem cells while adequately delivering drugs to cancer stem cells. Also, normal stem cells are environmentally sequestered thus will not be as reachable with drugs as will be cancer stem cells.

### Additional Supporting Evidence

The RNA-interference (RNA-I) mechanism has been shown to be active in normal mammalian stem cells and stem cells within cancers. Specifically, RNA-I functions in embryonic stem cells, and embryonic carcinoma cells as taught by the present application (Billy. Proc Natl Acad Sci 98:14428-14433, 2001; Paddison. Proc Natl Acad Sci 90:1443-1448, 2002).

Over expression of hiwi (a piwi homolog) was shown to have an anti-leukemic effect (Sharma. Blood 97:426-434, 2001) This application teaches that decreasing the expression of proteins, riboproteins in the fate determining mechanism results in an anticancer effect. Therefore, the teachings of this application are novel, and there is no conflict here. Also, this application also teaches that disruptions of noncoding nucleic acid interactions has an anti-cancer effect—something also not shown in this paper.

RNA-I has also been shown to be active in fast-growing cancer cells making up the tumor bulk, having inherited such from their cancer stem cell progenitor) in human cancers, specifically in prostate cancer cells (Lin. Biochem Biophys Res Commun 281:639-644, 2001).

RNA-I and other processes involving double-stranded RNA (dsRNA) (or the formation of such from single-stranded RNA or aberrant RNA) and the resolution thereof into processed forms (e.g. short RNA's) have indeed been found to be mechanistically related, as taught by the present application. More specifically, the term RNA-silencing (RNA-S) is now widely used to cover many of these related processes which include the formation of or recognition of or processing of or function of “aberrant RNA's”, microRNA's (miRNA's), antisense RNA's (asRNA's), short interfering RNA's (siRNA's), small temporal RNA's (stRNA's), et al (Grishok. Cell 106:23-34, 2001; Hutvagner. Science 293:834-838, 2001; Plasterk. Science 295:694, 2002; Land. Trends Genet 17:379, 2001; Lai. Nature Genet 30:363-364, 2002; Bender. Cell 106:129-132, 2001; Ambros. Cell 107:823-826, 2001; Moss. Curr Biol 12:R138-R140, 2002).

Hiwi, in inherited and sporadic forms, is involved in the genesis of stem cell-derived human testicular cancers (Qiao. Oncogene 21:3988-3999, 2002).

A host of human small RNA's (miRNA's) have indeed been reported, and are found to be expressed in a stage-specific (e.g. stem cell stage) and tissue-specific manner as taught by the present application (Pasquinelli. Trends Genet 18:171-173, 2002; Ruvkun. Science 294:797, 2001. Lagos-Quintana. Science 294:853-858, 2001; Lau. Science 294:858-862, 2001; Lee. Science 294:862-864, 2001).

The miRNA's, stRNA's, and other related species can be produced asymmetrically from a precursor RNA and are often single-stranded—thereby indicating strand-specificity so appear indeed to be linked to the leading-lagging concept (related to the asymmetric segregation of strands by stem cells) taught by the present application (Eddy. Nature Rev Genet 2:919-929, 2001).

As taught by the present application, snoRNA's can act not only on rRNA but other nucleic acid species (such as tRNA, DNA, et al) (Kiss. Cell 109:145-148, 2002, Maiorano. Exp Cell Res 252:165-174, 1999). Moreover, snoRNA-mediated modifications can be independent of or compete with other modifications such as RNA-I and RNA-editing (RNA-E) as explicitly taught by the present application (Scadden. EMBO Reports 2:1107-1111, 2001). This is key considering recent evidence of a role by snoRNA's in fate determination (e.g. at the IC-snurf-snrpn region wherein intron-encoded snoRNA's such as MBII-52 and MBII-85 reside) (Kiss. Cell 109:145-148, 2002). Also, as expected, certain snoRNA's have been implicated in cancer as evidence by documentation that the snoRNA, U50HG, resides at the B cell lymphoma translocation t(3;6(q27;q15) (Tanaka. Genes Cells 5:277-287, 2000).

As shown by recent experiments, processed RNA's of the type described by the present application can be very short (e.g. 20-25 bp) or slightly larger but still “short” thus the 20-25 bp size is not absolute, nor was it ever contemplated to be so by the present application (Huttenhofer. EMBO J 20:2943-2953, 2001; Eddy. Nature Rev Genet 2:919-929, 2001; Boutla. Nucl Acids Res 30:1688-1694, 2002). For example, an 85 bp RNA fraction has been described with RNA-I features/activity (Boutla. Nucl Acids Res 30:1688-1694, 2002). The point being that RNA's and dsRNA's are processed by RNA-I-type mechanisms into shorter fragments of varying lengths.

As taught by the present application, RNA-mediated DNA binding, DNA methylation, and chromatin alterations have indeed been described (Mette. EMBO J 19:5194-5201, 2000; Birchler. Curr Opin Genet Dev 10:211-216, 2000. Aravin. Curr Biol 11:1017-1027, 2001; Mette. EMBO J 18:241-248, 1999; Habu. Curr Opin Gene Dev 11:215-220, 2001; Pal Bhadra. Mol Cell 9:315-327, 2002. Matzke. Science 293:1080, 2001; Briggs. Nature Genet 30:241-242, 2002; Maison. Nature Genet 30:329-334, 2002). In humans, the BD transcript, on human chromosome 15q, binds DNA (Surani. Cell 93:309-312, 1998). The Air transcript, on human chromosome 11p15, controls imprinting regions also by RNA:DNA or RNA:RNA interactions (Sleutels. Nature 415:810-813, 2002). Moreover, a host of other noncoding RNA's appear to act in RNA:DNA and RNA:RNA interactions in humans/mammals (Erdmann. Nucl Acids Res 29:189-193, 2001).

Proteins involved in such RNA-mediated actions, i.e. chromomethylases and chromodomain proteins, have been described and are thus novel targets described by the present application (Martienssen. Science 293:1070-1074, 2001; Akhtar. Nature 407:405-409, 2000). Of note, a link between chromodomain proteins and strand-specific imprints (e.g. lagging strand) has indeed been described as taught by the present application (Nakayama. Cell 101:307-317, 2000). A key point being that noncoding RNA's (single- or double-stranded) can effect not only RNA-I (i.e. RNA degradation) but also DNA-level changes (e.g. methylation or changes in histones, histone acetylation, chromatin structure) as would occur if said RNA were transcribed from a promoter region—in this way, RNA would have homology to a DNA promoter region not a coding region. This has been described in transcriptional gene silencing (TGS) as compared with posttranscriptional gene silencing (PTGS), both of which occur not only in simple life forms, but in higher eukaryotes (including mammals/humans).

The experiments outlined by the present application are readily enabled. Namely, stem cell and rare cell purification techniques have been described (Miyazato. Blood 98:422-427, 2001; Kelly. Mol Reprod Dev 56:113-123, 2000; Iinuma. Int J Cancer 89:337-344, 2000; Yuasa. Cancer Lett 143:57-62, 1999). Also, techniques for the analysis of RNA from rare cells has been described (Klein. Nature Biotech 20:387-392, 2002). Moreover, techniques for the procurement or cloning of processed or small RNA's have also been performed (Elbashir. Genes Dev 15:188-200, 2001. Hutvagner. RNA 6:1445-1454, 2000. Djikeng. RNA 7:1522-1530, 2001).

The use of noncoding RNA's as targets within high throughput assays for drug screens is enabled. For example, assay systems measuring the effects upon noncoding RNA targets at the X-chromosome are enabled, e.g. because a variety of X-linked readout systems have been described (e.g. using detectable luciferase, fluorescence, et al) (Hadjantonakis. Genesis 29:133-140, 2001; Goto. Mol Reprod Dev 49:356-367, 1998).

Double-stranded RNA and small RNA's (i.e. miRNA's) have indeed been described in certain degenerative diseases, e.g. neurologic, in need of regenerative medicine therapies described by the present application (Michalowski. Nucl Acids Res 27:3534-3542, 1999; Mourelatos. Genes Dev 16:720-728, 2002).

A number of additional specific targets, not intended to be comprehensive but only to serve as as examples, described in full nature by the present application are:
Miwi, mili—mammalian piwi-related genes, mili expressed in stem cells of the germline (primordial germ cells) (Kuramochi-Miyagawa. Mech Dev 108:121-133, 2001);
Ago1—stem cell renewal (Arabidopsis, Drosophila) (Kataoka. Genes Cells 6:313-325, 2001);
alg-1, alg-2—nematode, heterochronic pathway (Grishok. Cell 106:23-34, 2001);
daf-12—affected by let-7 in nematodes, aging function (Reinhart. Nature 403:901-906, 2000);
human cgh-1 homolog (RCK/p54)—self-renewal, at 11q23 lymphoma translocation (Navarro. Development 128:3221-3232, 2001);
telomeres, centromeres, transposon sequences, rDNA, repeats, satellite sequences, retroelement sequences (Henikoff. Nature 417:227, 2002; Henikoff. Trends Genet 18:165-167, 2002;
Martienssen. Science 293:1070-1074, 2001);
Human Tsix (Migeon. Am J Hum Genet 69:951-960, 2001);
LIT1—imprinted antisense RNA at 11p15 (Mitsuya. Hum Mol Genet 8:1209-1217, 1999);
MSUD—meiotic silencing by unpaired DNA—proteins, unpaired nucleic acids (Cogoni. Nature Genet 30:245-246, 2002); IC-snurf-snrpn—and intron-encoded snoRNA's within (e.g. MBII-52, MBII-85); (Kiss. Cell 109:145-148, 2002; Runte. Hum Mol Genet 10:2687-2700, 2001);
snoRNA (U50HG) at B cell lymphoma translocation t(3;6)(q27;q15); (Tanaka. Genes Cells 5:277-287, 2000); human mir142, an stRNA, translocation in lymphomas (Moss. Curr Bio 12:R138, 2002);
a host of other noncoding RNA's that act in RNA:DNA and RNA:RNA interactions (Erdmann. Nucl Acids Res 29:189-193, 2001);
mitochondrial rRNA, mitochondrial tRNA BC1—small noncoding RNA, action in germ cell development (Muslimov. J Cell Sci 115:1243-1250, 2002);
Y RNA (Rutjes. J Biol Chem 274:24799-24807, 1999);
BD transcript, binds DNA, on human chromosome 15q (Surani. Cell 93:309-312, 1998);
KCNQ10T1/Kcnq1ot1 antisense transcripts flanked by LINE elements on human chromosome 11p15 (Engemann. Hum Mol Genet 9:2691-2706, 2000);
GNAS imprinted on human chromosome 20 (Aldred. Trends Genet 18:181, 2002);
DLK/GTL2 imprinted domain on human chromosome 14 (Wylie. Genome Res 10:1711-1718, 2000); other loci that undergo allelic exclusion and other loci harboring noncoding RNA's that are differentially regulated during rearing of progeny by stem cells.

### REFERENCES

1. Horvitz H R, Herskowitz I. Mechanisms of asymmetric cell division: two Bs or not two Bs, that is the question. Cell 68:237-255, 1992.
2. Jan Y N, Jan L Y. Asymmetric cell division. Nature 392:775-778, 1998.
3. Lin H, Schagat T. Neuroblasts: a model for the asymmetric division of stem cells. Trends Genet 13:33-39, 1997.
4. Hawkins N, Garriga G. Asymmetric cell division: from A to Z. Genes Dev 12:3625-3638, 1998.
5. Dalgaard J Z, Klar A J. Orientation of DNA replication establishes mating-type switching pattern in S. pombe. Nature 400:181-184, 1999.
6. Till J E. Stem cells in differentiation and neoplasia. J Cell Physiol Suppl 1:3-11, 1982
7. Colot V, Maloisel L, Rossignol J-L. Interchromosomal transfer of epigenetic states in ascobolus: transfer of DNA methylation is mechanistically related to homologous recombination. Cell 86:855-864.
8. LaSalle J M, Lalande M. Homologous association of oppositely imprinted chromosomal domains. Science 272: 725-728, 1996.
9. Steenman M J, Rainier S, Dobry C J, Grundy P, Horon I L, Feinberg A P. Loss of imprinting of IGF2 is linked to reduced expression and abnormal methylation of H19 in Wilms' tumour. Nat Genet 7:433-439, 1994.
10. Taniguchi T, Sullivan M J, Ogawa O, Reeve A E. Epigenetic changes encompassing the IGF2/H19 locus associated with relaxation of IGF2 imprinting and silencing of H19 in Wilms tumor. Proc Natl Acad Sci (USA) 92:2159-2163, 1995.
11. Hutvagner G, Mlynarova L, Nap J-P. Detailed characterization of the post-transcriptional gene-silencing-related small RNA in a GUS gene-silenced tobacco. RNA 6:1445-1454, 2000.
12. Elbashir S M, Lendeckel W, Tuschl T. RNA interference is mediated by 21- and 22-nucleotide RNAs. Genes Dev 15:188-200, 20001.
13. Marahrens Y. X-inactivation by chromosomal pairing events. Genes Dev 13:2624-2632, 1999.
14. Jones t, Hamilton A J, Voinnet O, Thomas C L, Maule A J, Baulcombe D C. RNA-DNA interactions and DNA methylation in post-transcriptional gene silencing. Plant Cell 11: 2291-2302, 1999.
15. Pelissier T, Wassenegger M. A DNA target of 30 bp is sufficient for RNA-directed DNA methylation. RNA 6:55-65, 2000.
16. Constancia M, Pickard B, Kelsey G, Reik W. Imprinting mechanisms. Genome Res 8:881-900, 1998
17. Fire A. RNA-triggered gene silencing. Trends Genet 15:358-363, 1999.
18. Birchler J A, Bhadra M P, Bhadra U. Making noise about silence: repression of repeated genes in animals. Curr Opin Gene Dev 10:211-216, 2000.
19. Wu C-t, Morris J R. Transvection and other homology effects. Curr Opin Gene Dev 9:237-246, 1999.
20. Bingham P M. Cosuppression comes to animals Cell 90:385-387, 1997.
21. Cogoni C, Macino G. Post-transcriptional gene silencing across kingdoms. Curr Opin Gene Dev 10:638-643, 2000.
22. Baulcombe D. Gene silencing: RNA makes RNA makes no protein. Curr Biol 9:R599-R601, 1999.
23. Tabara H, Sarkissian M, Kelly W G, Fleenor J, Grishok A, Timmons L, Fire A, Mello C C. The rde-1 gene, RNA interference, and transposon silencing in C. elegans. Cell 99:123-132, 1999.
24. Ketting R F, Haverkamp T H, van Luenen H G, Plasterk R H. Mut-7 of C. elegans, required for transposon silencing and RNA interference, is a homolog of Werner syndrome helicase and RNaseD. Cell 99:133-141, 1999.
25. Lyon M F. Imprinting and X-chromosome inactivation. Results Probl Cell Differ 25:73-90, 1999.
26. Szabo P E, Mann J R. Biallelic expression of imprinted genes in the mouse germ line: implications for erasure, establishment, and mechanisms of genomic imprinting. Genes Dev 9:1857-1868.
27. Feil R, Walter J, Allen J D, Reik W. Developmental control of allelic methylation in the imprinted mouse Igf2 and H19 genes. Development 120:2933-2943, 1994.
28. Kelsey G, Reik W. Imprint switch mechanism indicated by mutations in Prader-Willi and Angelman syndromes. Bioessays 19:361-365, 1997.
29. Ohlsson R, Tycko B, Sapienza C. Monoallelic expression: ‘there can only be one’. Trends Genet 14:435-438, 1998.
30. Rothenberg E V. Stepwise specification of lymphocyte developmental lineages. Curr Opin Gene Dev 10:370-379, 2000.
31. Monk M. Variation in epigenetic inheritance. Trends Genet 6:110-114, 1990.
32. Lee J T, Davidow L S, Warshawsky D. Tsix, a gene antisense to Xist at the X-inactivation centre. Nature Genet 21:400-404, 1999.
33. Kelsey G. The hows and whys of imprinting. Trends Genet 16:15-16, 2000.
34. Reaban M E, Griffin J A. Induction of RNA-stabilized DNA conformers by transcription of an immunoglobulin switch region. Nature 348:342-344, 1990.
35. Stavnezer J. A touch of antibody class. Science 288:984-985, 2000.
36. Daniels G A, Lieber M R. Strand specificity in the transcriptional targeting of recombination at immunoglobulin switch sequences. Proc Natl Acad Sci USA 92:5625-5629, 1995.
37. Harriman W, Volk H, Defranoux N, Wabl M. Immunoglobulin class switch recombination. Ann Rev Immunol 11:361-384, 1993.
38. Panning B, Jaenisch R. RNA and the epigenetic regulation of X chromosome inactivation. Cell 93:305-308, 1998.
39. Debrand E, Chureau C, Arnaud D, Avner P, Heard E. Functional analysis of the DXPas34 locus, a 3′ regulator of Xist expression. Mol Cell Biol 19:8513-8525, 1999.
40. Mise N, Goto Y, Nakajima N, Takagi N. Molecular cloning of antisense transcripts of the mouse Xist gene. Biochem Biophys Res Comm 258:537-541, 1999.
41. Wagner E G H. Antisense RNA control in bacteria, phages, and plasmids. Ann Rev Microbiol 48:713-742, 1994.
42. Cedar H, Bergman Y. Developmental regulation of immune system gene rearrangement. Curr Opin Immunol 11:64-69, 1999.
43. Hayakawa K, Hardy R R. Development and function of B-1 cells. Curr Opin Immunol 12:346-353, 2000.
44. Qin X-F, Schwers S, Yu W, Papavasiliou F, Suh H, Nussenzweig A, Rajewsky K, Nussenzweig M C. Secondary V(D)J recombination in B-1 cells. Nature 397:355-359, 1999.
45. Nutt S L, Busslinger M. Monoallelic expression of Pax5: A paradigm for the haploinsufficiency of mammalian Pax genes? Biol Chem 380:601-611, 1999.
46. Mostoslavsky R, Singh N, Kirillov A, Pelanda R, Cedar H, Chess A, Bergman Y. K chain monoallelic demethylation and the establishment of allelic exclusion. Genes Dev 12:1801-1811, 1998.
47. Finnegan E J, Wang M-B, Waterhouse P. Gene silencing: Fleshing out the bones. Curr Biol 11:R99-R102, 2001.
48. Webber A L, Ingram R S, Levorse J M, Tilghman S M. Location of enhancers is essential for the imprinting of H19 and Igf2 genes. Nature 391:711715, 1998.
49. Reizis B, Lee J T, Leder P. Homologous genomic fragments in the mouse pre-T cell receptor alphas (pTa) and Xist loci. Genomics 63:149-152, 2000.
50. Saksela K, Baltimore D. Negative regulation of immunoglobulin kappa light-chain gene transcription by a short sequence homologous to the murine B1 repetitive element. Mol Cell Biol 13:3698-3705, 1993.
51. Fiering S, Whitelaw E, Martin D I K. To be or not to be active: the stochastic nature of enhancer action. Bioessays 22:381-387, 2000.
52. Teng S C, Kim B, Gabriel A. Retrotransposon reverse-transcriptase-mediated repair of chromosomal breaks. Nature 383:641-644, 1996.
53. Heyman T, Agoutin B, Friant S, Wilhem F X, Wilhelm M L. Plus-strand DNA synthesis of the yeast retrotransposon Tyl is initiated at two sites, PPT1 next to the 3′ LTR and PPT2 within the pol gene. PPT1 is sufficient for Tyl transposition. J Mol Biol 253:291-303, 1995.
54. Cairns J. Mutation selection and the natural history of cancer. Nature 255:197-200, 1975.
55. Lauermann V. DNA repair by recycling reverse transcripts. Nature 386:31-32, 1997.
56. Muller J R, Marcu K B. Stimulation of murine B lymphocytes induces a DNA exonuclease whose activity on switch-u DNA is specifically inhibited by other germ-line switch region RNAs. J Immunol 160:3337-3341, 1998.
57. Lund A H, Duch M, Pedersen F S. Selection of functional tRNA primers and primer binding site sequences from a retroviral combinatorial library: identification of new functional tRNA primers in muring leukemia virus replication. Nucleid Acids Res 28:791-799, 2000.
58. Lyon M F. X-chromosome inactivation: a repeat hypothesis. Cytogenet Cell Genet 80:133-137, 1998.
59. Wrobel B, Wegrzyn G. Replication regulation of ColE1-like plasmids in amino acid-starved Escherichia coli. Plasmid 39:48-62, 1998.
60. Yavachev L, Ivanov I. What does the homology between E. coli tRNAs and RNAs controlling ColE1 plasmid replication mean? J Theor Biol 131:235-241, 1988.
61. Tollervey D, Kiss T. Function and synthesis of small nucleolar RNAs. Curr Opin Cell Biol 9:337-342, 1997.
62. Omer A D, Lowe T M, Russell A G, Ebhardt H, Eddy S, Dennis P P. Homologs of small nucleolar RNAs in Archaea. Science 288:517-522, 2000.
63. Filipowicz W. Imprinted expression of small nucleolar RNAs in brain: Time for RNomics. Proc. Natl Acad Sci USA 97:14035-14037, 2000.
64. Cavaille J, Buiting K, Kiefmann, Lalande M, Brannan C I, Hortsthemke B, Bachellerie J-P, Brosius J, Huttenhofer A. Identification of brain-specific and imprinted small nucleolar RNA genes exhibiting an unusual genomic organization. Proc Natl Acad Sci USA 97:14311-14316, 2000.
65. Keeney J B, Chapman K B, Lauermann V, Voytas D F, Astrom S U, von Pawel-Rammingen U, Bystrom A, Boeke J D. Multiple molecular determinants for retrotransposition in a primer tRNA. Mol Cell Biol 15:217-226, 1995.
66. Kiss-Laszlo Z, Henry Y, Bachellerie J P, Caizergues-Ferrer M, Kiss T. Site-specific ribose methylation of preribosomal RNA: a novel function for small nucleolar RNAs. Cell 85:1077-1088, 1996.
66A. Cavaille J, Nicoloso M, Bachellerie J P. Targeted ribose methylation of RNA in vivo directed by tailored antisense RNA guides. Nature 383:732-735, 1996.
67. Csuka I, Banfalvi G. Analysis of 5′-termini of early intermediates of okazaki fragments accumulated in thymocytes after emetine treatment of mice. DNA Cell Biol 16:979-984, 1997.
67A. Nethanel T, Reisfeld S, Dinter-Gottlieb G, Kaufmann G. An okazaki piece of simian virus 40 may be synthesized by ligation of shorter precursor chains. J Virol 62:2867-2873, 1988.
68. Rodriguez I R, Mazuruk K, Jaworski C, Iwata F, Moreira E F, Kaiser-Kupfer M I. Novel mutations in the XLRS1 gene may be caused by early okazaki fragment sequence replacement. Invest Ophthal Vis Sci 39:1736-1739, 1998.
69. Clayton D A. A big development for a small RNA. Nature 410:29-31, 2001.
70. Ridanpaa M, van Eeneennam K, Pelin K, Chadwick R, Johnson C, Yuan B, vanVenrooij W, Pruijn G, Salmela R, Rockas S, Makitie O, Kaitila I, de la Chapelle A. Mutations in the RNA component of RNase MRP cause a pleiotropic human disease, cartilage-hair hypoplasia. Cell 104:195-203, 2001.
70A. Jen K-Y, Gewirtz A M. Suppression of gene expression by targeted disruption of messenger RNA: available options and current strategies. Stem Cells 18:307-319, 2000.
71. Bonin M, Oberstrab J, Lukacs N, Ewert K, Oesterschulze, E, Kassing R, Nellen W. Determination of preferential binding sites for anti-dsRNA antibodies on double-stranded RNA by scanning for microscopy. RNA 6:563-570, 2000.
72. White L K, Wright W E, Shay J W. Telomerase inhibitors. Trends Biotechnol 19:114-120, 2001.
73. Xavier K A, Eder P S, Giordano T. RNA as a drug target: methods for biophysical characterization and screening. Trends Biotechnol 18:349-356, 2000.
74. Ecker D J, Griffey R H. RNA as a small-molecule drug target: doubling the value of genomics. Drug Discov Today 4:420-429, 1999.
75. Hermann T, Westhof E. Rational drug design and high-throughput techniques for RNA targets. Comb Chem High Throughput Screen 3:219-234, 2000.
76. Sucheck S J, Wong C-H. RNA as a target for small molecules. Curr Opin Chem Biol 4:678-686, 2000.
77. Hines P J, Purnell B A, Marx J. Stem cells branch out. Science 287:1417, 2000.
77A. Pierce G B, Speers W C. Tumors as caricatures of the process of tissue renewal: prospects for therapy by directing differentiation. Cancer Res 48:1996-2004, 1988.
78. Taylor M F, Wiederholt K, Sverdrup F. Antisense oligonucleotides: a systematic high-throughput approach to target validation and gene function determination. Drug Discov Today 4:562-567, 1999.
79. Hadjantonakis A K, Cox L L, Tam P P, Nagy A. An X-linked GFP transgene reveals unexpected paternal X-chromosome activity in trophoblastic giant cells of the mouse placenta. Genesis 29:133-140, 2001.
80. Ainscough J F, Koide T, Tada M, Barton S, Surani M A. Imprinting of Igf2 and H19 from a 130 kb YAC transgene. Development 124:3621-3632, 1997
81. Kozian D H, Kirschbaum B J. Comparative gene-expression analysis. Trends Biotechnol 17:73-78, 1999.
82. Mass S, Melcher T, Seeburg P H. Mammalian RNA-dependent deaminases and edited mRNAs. Curr Opin Cell Biol 9:343-349, 1997.
83. Kumar M, Carmichael G G. Antisense RNA: function and fate of duplex RNA in cells of higher eukaryotes. Microbiol Molec Bio Rev 62:1415-1434, 1998.
84. Kumar M, Carmichael G G. Nuclear antisense RNA induces extensive adenosine modifications and nuclear retention of target transcripts. Proc Natl Acad Sci USA 94:3542-3547, 1997.
85. Hamilton A J, Baulcombe D C. A species of small antisense RNA in postranscriptional gene silencing in plants. Science 286:950-952, 1999.
86. Zamore P D, Tuschl T, Sharp P A, Bartel D P. RNAi: double-stranded RNA directs the ATP-dependent cleavage of mRNA at 21 to 23 nucleotide intervals.
87. Parrish S, Fleenor J, Xu S, Mello C, Fire A. Functional anatomy of a dsRNA trigger: differential requirement for the two trigger strands in RNA interference. Mol Cell 6:1077-1087, 2000.
88.
89. Tracy R B, Hsieh C-L, Lieber M R. Stable RNA/DNA hybrids in the mammalian genome: inducible intermediates in immunoglobulin class switch recombination. Science 288:1058-1061, 2000.
90. Proud C. PKR: a new name and new roles. Trends Biochem Sci 20:241-246, 1995.
91. Kidner C. Initiating interference. Trends Genet 17:129, 2000.
92. Baulcombe D. Diced defense. Nature 409:295296, 2001.
93. Bernstein E, Caudy A A, Hammond S M, Hannon G J. Role for a bidentate ribonuclease in the initiation step of RNA interference. Nature 409:363-366, 2001.
94. Askew D S, Xu F. New insights into the function of noncoding RNA and its potential role in disease pathogenesis. Histol Histopathol 14:235-241, 1999.
95. Moss E G. Non-coding RNAs: lightning strikes twice. Curr Biol 10:R436-R439, 2000.
96. Eddy S R. Noncoding RNA genes. Curr Opin Genet Dev 9:695-699, 1999. noncoding RNAs
97. Sleutels F, Barlow D P, Lyle R. The uniqueness of the imprinting mechanism. Curr Opin Gene Dev 10:229-233, 2000.
98. Kelsey G. The hows and whys of imprinting. Trends Genet 16:15-16, 2000.
98A. Sijen T, Vijn I, Rebocho A, van Blokland R, Roelofs D, Mol J N M, Kooter J M. Transcriptional and post-transcriptional gene silencing are mechanistically related. Curr Biol 11:436-440, 2001.
99. Lakhotia S C. RNA polymerase 11 dependent genes that do not code for protein. Ind J Biochem Biophys 33:93-102, 1996.
100. Jurka J. Repbase update a database and an electronic journal of repetitive elements. Trends Genet 16:418-420, 2000.
101. Gu J, Reddy R. Small RNA database. Nucleic Acids Res 26:160-162, 1998.
102. Sprinzl M, Horn C, Brown M, Ioudovitch A, Steinberg S. Compilation of tRNA sequences and sequences of tRNA genes. Nucleic Acids Res 26:148-153, 1998.
103. Helm M, Brule H, Friede D, Giege R, Putz D, Florentz C. Search for characteristic structural features of mammalian mitochondrial tRNAs. RNA 6:1356-1379, 2000.
104. Tollervey D, Kiss T. Function and synthesis of small nucleolar RNAs. Curr Opin Cell Biol 9:337-342, 1997.
105. Peculis B. RNA-binding proteins: if it looks like a sn(o)RNA . . . . Curr Biol 10:R916-R918, 2000.
106. Gaspin C, Cavaille J, Erauso G, Bachellerie J P. Archael homology of eukaryotic methylation guide small nucleolar RNAs: lessons from the Pyrococcus genomes. J Mol Biol 297:895-906, 2000.
107. Erdmann V A, Szymanski M, Hochberg A, Groot N, Barciszewski J. Non-coding, mRNA-like RNAs database Y2K. Nucleic Acids Res 28:197-200, 2000.
108. Szymanski M, Barciszewska J, Barciszewski J, Erdmann V A, 5S ribosomal RNA database Y2k. Nucleic Acids Res 28:166-167, 2000.
109. Adoutte A. Small but mighty timekeepers. Nature 408:37-38, 2000.
110. Pasquinelli A E, Reinhart B J, Slack F, Martindale M Q, Kuroda M I, Maller B, Hayward D C, Ball E E, Degnan B, Muller P, Spring J, Srinivasan A, Fishman M, Finnerty J, Corbo J, Levine M, Leahy P, Davidson E, Ruvkun G. Conservation of the sequence and temporal expression of let-7 heterochronic regulatory RNA. Nature 408:86-89, 2000.
111. Millar J K, Wilson-Annan J C, Anderson S, Christie S, Taylor M S, Semple C A M, Devon R S, St Clair D M, Muir W J, Blackwood D H R, Porteous D J. Disruption of two novel genes by translocation co-segregating with schizophrenia. Hum Molec Genet 9:1415-1423, 2000.
112. Morelli C, Magnanini C, Mungall A J, Negrini M, Barbanti-Brodano G. Cloning and characterization of two overlapping genes in a subregion at 6q21 involved in replicative senscence and schizophrenia. Gene 252:217-225, 2000.
116. Mirochnitchenko O, Inouye M. Evaluation of the use of antisense tRNA(met) as an inhibitor for eukaryotic protein synthesis. Antisense Res Dev 3:171-179, 1993.
117. Utz E D, Siravo-Sagraves G M, Trewyn R W, Morgan C J. Detection of human tRNAs with antisense oligonucleotides. Anal Biochem 216:110-117, 1994.
118. Cavaille J, Nicoloso M, Bachellerie J P. Targeted ribose methylation of RNA in vivo directed by tailored antisense RNA guides. Nature 383:732-735, 1996.
119. Kiss-Laszlo Z, Henry Y, Bachellerie J P, Caizergues-Ferrer M, Kiss T. Site-specific ribose methylation of preribosomal RNA: a novel function for small nucleolar RNAs. Cell 85:1077-1088, 1996.
120. Chaganti R S K, Houldsworth J. The cytogenetic theory of the pathogenesis of human adult male germ cell tumors. APMIS 106:80-84, 1998.
121. Sinke R J, Van Asseldonk M, De Bruijn D, Strijk J A, Merkx G, Weghuis D O, De Jong B, Oosterhuis J W, Van Kessel A G. Towards the isolation of a human malignant extragonadal germ cell tumour-associated breakpoint in chromosome 11q13. APMIS 106:73-79, 1998.
122. Jost J-P, Fremont M, Siegmann M, Hofsteenge J. The RNA moiety of chick embryo 5-methylcytosine-DNA glycosylase targets DNA demethylation. Nucl Acids Res 25:4545-4550, 1997.
123. Neuberger M S, Scott J. RNA editing aids antibody diversification. Science 289:1075-1076, 2000.
124. Tian M, Alt F W. RNA editing meets DNA shuffling. Nature 407:31-33, 2000.
125. Looijenga L H J, Gillis A J M, van Gurp R J H L M, Verkerk A J M H, Oosterhuis J W. X inactivation in human testicular tumors. Am J Pathol 151: 581-590, 1997.
126. Looijenga L H J, Verkerk A J M H, Dekker M C, van Gurp R J H L M, Gillis A J M, Oosterhuis J W. Genomic imprinting in testicular germ cell tumors. APMIS 106: 187-197, 1998.
127. Feinberg A P. Imprinting of a genomic domain of 11p15 and loss of imprinting in cancer: an introduction. Cancer Res (Suppl) 59: 1743s-1746s, 1999.
128. Squire J, Weksberg R. Genomic imprinting in tumors. Semin Cancer Biol 7: 41-47, 1996.
129. Looijenga L H J, Verkerk A J M H, De Groot N, Hochberg A A, Oosterhuis J W. H19 in normal development and neoplasia. Molec Reprod Dev 46: 419-439, 1997.
130. Lee J T. Disruption of imprinted X inactivation by parent-of-origin effects of Tsix. Cell 103: 17-27, 2000.
131. Lee J T, Lu N. Targeted mutagenesis of Tsix leads to nonrandom X inactivation. Cell 99: 47-57, 1999.
132. Cairns J. Mutation selection and the natural history of cancer. Nature 255:197-200, 1975.
133. Dalgaard J Z, Klar A J. Orientation of DNA replication establishes mating-type switching pattern in S. pombe. Nature 400:181-184, 1999.
134. Kelsey G. The hows and whys of imprinting. Trends Genet 16:15-16, 2000.
135. Matzke M A, Matzke A J M, Pruss G J, Vance V B. RNA-based silencing strategies in plants. Curr Opin Gene Dev 11: 221-227, 2001.
136. Vaucheret H, Fagard M. Transcriptional gene silencing in plants: targets, inducers, and regulators. Trends Genet 17: 29-35, 2001.
137. Monk M. Variation in epigenetic inheritance. Trends Genet 6: 110-114, 1990.
138. Mostoslavsky R, Singh N, Kirillov A, Pelanda R, Cedar H, Chess A, Bergman Y. K chain monoallelic demethylation and the establishment of allelic exclusion. Genes Dev 12: 1801-1811, 1998.
139. Bass B L. RNA interference: The short answer. Nature 411: 428-429, 2001.
140. Elbashir S M, Harborth J, Lendeckel W, Yalcin A, Weber K, Tuschl T. Duplexes of 21-nucleotide RNAs mediate RNA interference in cultured mammalian cells. Nature 411: 494-498, 2001.

What is claimed is:

1. A method of increasing desirable cell population or decreasing undesirable cell population which comprises manipulating the fate of a cell through contact with a compound that affects a cell fate-determining mechanism involving RNA-silencing, RNA-interference, RNA-editing, snoRNA-mediated modifications, tRNA primed events, or other fate-determining mechanism involving homologous nucleic acid interactions of RNA:RNA, RNA:DNA, or DNA:DNA and their resolution under conditions to cause the cell to change or maintain fate.

2. The method of claim 1, wherein the change or maintenance of cell fate results in cell regeneration, cell differentiation, or cell death for applications involving regenerative biology, medicine, developmental biology, cancer, and aging.

3. The method of claim 1, wherein the cell is a stem cell, regenerative cell, or a cancer cell and wherein the compound is naturally occurring, synthesized, or procured through a screen and comprises a nucleic acid, a protein, a riboprotein, a vaccine, a small molecule, or a chemical compound.

4. The method of claim 3, wherein the compound is identified through use of an assay in a screen of biological or chemical libraries of compounds.

5. The method of claim 3, wherein the compound is intelligently designed.

6. The method of claim 1, wherein the compound affects a cell fate-determining untranslated/noncoding RNA species (cuR),or a cuR binding DNA region.

7. The method of claim 6, wherein the cuR is a sense-cuR or an anti-cuR.

8. The method of claim 6, wherein the cuR is single-stranded, double-stranded, part of an RNA:DNA complex, or part of a riboprotein complex.

9. The method of claim 6, wherein the cuR is modified by RNA-silencing, RNA-interference, RNA-editing, or snoRNA-mediated modification.

10. The method of claim 9, wherein the modified cuR is a small interfering RNA, small temporal RNA, microRNA, aberrant RNA, edited RNA, guide RNA, tRNA, tRNA-like species, snoRNA, snoRNA-like species, retroelement-derived RNA, imprinted RNA, allelically excluded RNA, or other modified or noncoding RNA.

11. The method of claim 6, wherein the cuR is one of the following: Tsix, Xist, a switch (S) sequence, let-7, lin-4, kappaNE, kappaBS, Disc1, Disc2, C6orf4-6, C6UAS, Okazaki RNA, tRNA, tRNA-like species, snoRNA, snoRNA-like species, Air RNA, BC1 RNA, mir 142, or noncoding RNA within regions i(12p), 11q13, 11p, 15q.

12. The method of claim 1, wherein the compound is administered to a human in order to increase desirable cell population or decrease undesirable cell population therein.

13. The method of claim 12, wherein the compound is administered in combination with another related compound, or agent including chemotherapy, radiation, differentiation, immunotherapy, gene therapy, cancer therapy, or regenerative therapy.

14. The method of claim 13, wherein the administration causes a level of toxicity which is clinically tolerable.

15. The method of claim 1, wherein the compound affects a cuR-associated protein, a cuR-associated riboprotein, or other nucleic acid, protein or riboprotein involved in the cell fate-determining mechanism.

16. The method of claim 15, wherein the protein or riboprotein is qde1, sgs2, ego1, qde2, rde1, qde3, mut7, piwi, hiwi, MRP, or ADAR.

17. A method of manipulating the fate of a cell, which comprises contacting the cell with at least one of (a) a cell fate-determining untranslated/noncoding RNA species (cuR), (b) a modified cuR, or (c) a compound that modifies or affects cuR, under conditions sufficient to cause a cell-changing or cell-maintaining fate that results in cell regeneration, cell differentiation or cell death, so that an increase of desirable cells or a decrease in undesirable cells can be obtained.

18. A method of procuring a cell fate-determining substance from a stem cell or progenitor cell for use as a target or a compound which comprises:
purifying stem cells or progenitor cells from a larger population of cells;
isolating cell fate-determining untranslated/noncoding RNA species (cuRs) which includes modified cuRs from the stem cell or progenitor cells, optionally with amplification, if necessary, to form an isolate;
comparing the isolate with cuRs of non-stem cells to identify stem cell-specific cuRs; and
identifying the stem cell-specific cuRs by one of: (a) cloning and sequencing; (b) screening the stem cell-specific cuRs for cell fate maintaining or cell fate changing activity through use of an assay system; or (c) using the stem cell-specific cuRs as probes to screen a library of compounds for complementarity or cell fate maintaining or cell fate changing activity through use of an assay system.

19. The method of claim 18, wherein cancer stem cells are purified from blood, bone marrow, or tissues of patients, cuRs are isolated, and if necessary amplified, from the cancer stem cells, the cuRs are compared by subtraction or differential analysis with cuRs from non-stem cells, the cuRs are cloned, the library of cuRs is screened for activity, the library of cuRs is used to screen a cDNA array or other collection of nucleic acids, or the cuRs are sequenced and identified.

20. A therapeutic method of treating a condition in a subject by manipulating a fate-determining mechanism in a cell in the subject which comprises administering to the subject an effective amount of a compound that inhibits, maintains or activates a cell fate-determining mechanism, wherein the compound includes at least one of (a) a cell fate-determining untranslated/noncoding RNA species (cuR), (b) a modified cuR, or (c) a compound that modifies or affects cuR, under conditions sufficient to cause a cell changing or cell-maintaining fate that results in cell regeneration, cell differentiation or cell death, so that an increase of desirable cells or a decrease in undesirable cells can be obtained in the subject to assist in treating the condition.

21. A method of decreasing undesirable cell populations, as for cancer treatment, which comprises manipulating the mechanism involving RNA-silencing, RNA-interference, RNA-editing, snoRNA-mediated modifications, tRNA-primed events, and other homologous nucleic acid interactions of RNA:RNA, RNA:DNA, DNA:DNA and their resolution under conditions to cause the cell to change fate by virtue of activating parasitic elements including endogenous viruses, transposons, and related selfish elements.
